(19) United States
(12) Patent Application Publication
(10) Pub. No.: US 2021/0315468 A1
(43) Pub. Date: Oct. 14, 2021

(54) INFORMATION PROCESSING APPARATUS, INFORMATION PROCESSING METHOD, AND PROGRAM

(71) Applicant: SONY CORPORATION, TOKYO (JP)

(72) Inventor: TAKASHI FUJIMOTO, TOKYO (JP)

(21) Appl. No.: 17/284,563

(22) PCT Filed: Oct. 10, 2019
(86) PCT No.: PCT/JP2019/039970

§ 371 Date: Apr. 12, 2021

(30) Foreign Application Priority Data
Oct. 19, 2018 (JP) 2018-197354

(51) Int. Cl. A61B 5/0205; A61B 5/024; A61B 5/021; A61B 5/08; A61B 5/11; A61B 5/369; A61B 5/389; A61B 5/145; A61B 5/00

(52) U.S. Cl. CPC A61B 5/02055; A61B 5/02438; A61B 5/021; A61B 5/0816; A61B 5/11; A61B 2562/0219; A61B 5/389; A61B 5/14542; A61B 5/6801; A61B 5/742; A61B 2562/0271; A61B 5/369

(57) ABSTRACT

To provide an information processing apparatus, an information processing method, and a program that can provide a time for an individual according to how the individual feels the flow of time. Provided is an information processing apparatus including: an information acquisition unit (320) that acquires a temporal change in biological information from one or a plurality of biological information sensors worn by a user; and a calculation unit (332) that calculates a difference between a temporal change in first biological information in a first section and a temporal change in second biological information in a second section having a same time as the first section at predetermined time intervals and calculates a time difference with respect to a standard time.

### TECHNICAL FIELD

The present disclosure relates to an information processing apparatus, an information processing method, and a program.

### BACKGROUND ART

Many people live using time (e.g., standard time (standard time point)) based on atomic time determined by an atomic clock that keeps time by using a transition between specific energy levels, such as an atom, as an oscillator.

### CITATION LIST

### Patent Document

Patent Document 1: Japanese Patent Application Laid-Open No. 2005-13385

### SUMMARY OF THE INVENTION

### Problems to be Solved by the Invention

However, there is a pace of (progress) of flow of time according to each person's situation. In other words, the way people feel the flow of time varies with the situation of each person. Then, in addition to individual attributes (gender, age, and the like), such a difference in feeling changes depending on many factors such as the amount of physical exercise, amount of burden, physical conditions, and mental state of the day.

Therefore, the present disclosure proposes an example of an information processing apparatus, an information processing method, and a program that can provide a time for an individual according to how the individual feels the flow of time.

### Solutions to Problems

According to the present disclosure, there is provided an information processing apparatus including: an information acquisition unit that acquires a temporal change in biological information from one or a plurality of biological information sensors worn by a user; and a calculation unit that calculates a difference between a temporal change in first biological information in a first section and a temporal change in second biological information in a second section having a same time as the first section at predetermined time intervals and calculates a time difference with respect to a standard time.

Furthermore, according to the present disclosure, there is provided an information processing method including: acquiring a temporal change in biological information from one or a plurality of biological information sensors worn by a user; and calculating a difference between a temporal change in first biological information in a first section and a temporal change in second biological information in a second section having a same time as the first section at predetermined time intervals and calculating a time difference with respect to a standard time.

Moreover, according to the present disclosure, there is provided a program for causing a computer to execute: a function of acquiring a temporal change in biological information from one or a plurality of biological information sensors worn by a user; and a function of calculating a difference between a temporal change in first biological information in a first section and a temporal change in second biological information in a second section having a same time as the first section at predetermined time intervals and calculating a time difference with respect to a standard time.

### Effects of the Invention

According to the present disclosure described above, it is possible to provide a time for an individual according to how the individual feels the flow of time.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram (No. 1) for explaining a concept of an embodiment of the present disclosure.

FIG. 2 is an explanatory diagram (No. 1) for explaining an example of calculation of user time 412 in an embodiment of the present disclosure.

FIG. 3 is an explanatory diagram (No. 2) for explaining an example of calculation of user time 412 in an embodiment of the present disclosure.

FIG. 4 is an explanatory diagram (No. 2) for explaining a concept of an embodiment of the present disclosure.

FIG. 5 is an explanatory diagram for explaining an example of a configuration of an information processing system 1 according to an embodiment of the present disclosure.

FIG. 6 is a block diagram showing an example of a configuration of a wearable device 10 according to an embodiment of the present disclosure.

FIG. 7 is an explanatory diagram for explaining an example of the appearance of the wearable device 10 according to an embodiment of the present disclosure.

FIG. 8 is a block diagram showing an example of a configuration of a server 30 according to an embodiment of the present disclosure.

FIG. 9 is a flowchart showing an example of an information processing method according to an embodiment of the present disclosure.

FIG. 10 is an explanatory diagram for explaining an example of a display screen 800a according to an embodiment of the present disclosure.

FIG. 11 is an explanatory diagram for explaining an example of a display screen 800b according to an embodiment of the present disclosure.

FIG. 12 is an explanatory diagram for explaining an example of a display screen 800c according to an embodiment of the present disclosure.

FIG. 13 is an explanatory diagram for explaining an example of a display screen 800d according to an embodiment of the present disclosure.

FIG. 14 is an explanatory diagram for explaining an example of a display screen 800e according to an embodiment of the present disclosure.

FIG. 15 is an explanatory diagram for explaining an example of a display screen 800f according to an embodiment of the present disclosure.

FIG. 16 is an explanatory diagram for explaining an example of a display screen 800g according to an embodiment of the present disclosure.

FIG. 17 is an explanatory diagram for explaining an example of a display screen 800h according to an embodiment of the present disclosure.

FIG. 18 is an explanatory diagram for explaining an example of a display screen 850a according to an embodiment of the present disclosure.

FIG. 19 is an explanatory diagram for explaining an example of a display screen 850b according to an embodiment of the present disclosure.

FIG. 20 is an explanatory diagram for explaining an example of a display screen 850c according to an embodiment of the present disclosure.

FIG. 21 is an explanatory diagram for explaining an example of a display timing according to an embodiment of the present disclosure.

FIG. 22 is an explanatory diagram for explaining an example of a transition of a calculation mode according to an embodiment of the present disclosure.

FIG. 23 is a flowchart showing an example of an information processing method in an automatic mode according to an embodiment of the present disclosure.

FIG. 24 is a flowchart showing an example of processing for selecting reference data 420 according to an embodiment of the present disclosure.

FIG. 25 is an explanatory diagram for explaining an example of a display screen 850d according to an embodiment of the present disclosure.

FIG. 26 is a block diagram showing an example of a hardware configuration of an information processing apparatus 900 according to one embodiment of the present disclosure.

### MODE FOR CARRYING OUT THE INVENTION

A preferred embodiment of the present disclosure will be described in detail below with reference to the accompanying drawings. Note that, in the present specification and the drawings, configuration elements that have substantially the same function and configuration are denoted with the same reference numerals, and repeated description is omitted.

Furthermore, in this specification and the drawings, multiple configuration elements that have substantially the same or similar function and configuration can be denoted with the same symbols followed by different numerals to be distinguished. However, in a case where there is no need in particular to distinguish a plurality of configuration elements that has substantially the same or similar function and configuration, the same symbol alone is attached. Furthermore, similar configuration elements in a different embodiment can be distinguished by being designated with different alphabets after the same symbol. However, in a case where there is no particular need to distinguish between similar configuration elements, only the same reference numerals will be given.

Note that the description is given in the order below.

1. Background to the creation of the embodiment of the present disclosure
1.1. Background
1.2. Concept

2. Embodiment of the present disclosure
2.1. Configuration of an information processing system 1 according to the embodiment of the present disclosure
2.2. Configuration of a wearable device 10 according to the embodiment of the present disclosure
2.3. Configuration of a server 30 according to the embodiment of the present disclosure
2.4. Information processing method according to the embodiment of the present disclosure
2.5. Setting of start time according to the embodiment of the present disclosure
2.6. Presentation method according to the embodiment of the present disclosure
2.7. Timing of presentation according to the embodiment of the present disclosure
2.8. Timing of calculation according to the embodiment of the present disclosure
2.9. Selection of reference data 420 according to the embodiment of the present disclosure
2.10. Feedback processing of user evaluation according to the embodiment of the present disclosure
2.11. Example of using a user interface according to the embodiment of the present disclosure

3. Examples according to the embodiment of the present disclosure
3.1. Example 1
3.2. Example 2

4. Conclusion

5. Regarding hardware configuration

6. Supplement

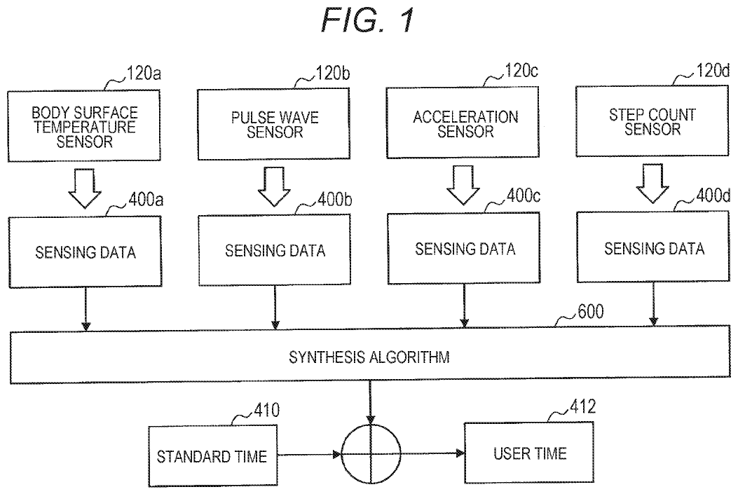

Note that, in the following description, a person who is equipped with the wearable device 10 (see FIG. 1) according to the embodiment of the present disclosure described below is referred to as the user.

### 1. Background to the Creation of the Embodiment of the Present Disclosure

### 1.1. Background

First, before describing the details of the embodiment of the present disclosure, the background leading to the creation of the embodiment of the present disclosure by the present inventor will be described.

Usually, “time interval” is defined as the length between two points in the flow of time, and “time point” is defined as the moment (one point) in the flow of time. As described earlier, many people live using time (e.g., standard time (standard time point) 410 (see FIG. 1)) based on atomic time determined by an atomic clock. In other words, the daily lives of many people are dominated by standard time 410.

However, daily life does not seem to progress steadily according to the progress of time of the standard time 410, but in some cases seems to be felt to be faster or slower according to the progress of time that changes according to the individual situation. For example, on days when a person spent a lot of time relaxing, the person often feels that “it was a long day today.” On the other hand, on days when the person was busy, the person often feels that “It's already such a time. Today was quick.” In other words, how the individual feels the progress of time changes every day depending on the individual situation.

Therefore, the present inventor, on the basis of the above-mentioned actual feelings in daily life, conducted a thought experiment to see what kind of influences can be given to the individual life in a case where the time for an individual according to how the individual feels the progress of time (in the following description, “user time 412 (see FIG. 1)”) is provided instead of the standard time 410.

For example, a case where the user time 412 is 11:00 pm even though it is 9:00 pm in the standard time 410, that is, a case where the progress of time of the user time 412 is faster than the progress of time of the standard time 410 is considered. The individual provided with the user time 412 as described above realizes that the progress of time has become faster because it was a busy working day. Then, the individual recognizes that he or she is tired from busy working, and selects an action of going to bed earlier than 11:00 pm in the standard time 410 even though he or she usually goes to bed at 11:00 pm in the standard time 410. That is, the present inventor has considered that by providing the user time 412 as described above, the user is released from the control from the standard time 410, which in turn leads to arousing the action change of the individual. Moreover, the present inventor has considered that if the individual can appropriately take an action according to the user time 412, it can lead to the maintenance of the health of the individual.

Therefore, the present inventor has diligently studied the method of calculating the user time 412 in order to provide many people with a time released from the standard time 410, that is, a time “user time 412” for the individual according to how the individual feels the progress of time.

There are various factors that influence how the individual feels the progress of time. Examples of the above-mentioned factors include individual attributes (gender, age, and the like), the amount of physical exercise, amount of burden, and mental state (relaxed state) of the day. Therefore, the present inventor has considered that the user time 412 can be calculated by estimating changes in how the individual feels the progress of time by paying attention to the amount of physical exercise and the amount of burden of the day of the individual among the above-mentioned factors. In detail, the present inventor has considered that, among the above-mentioned factors, the individual attributes are not factors that significantly change every day, and therefore the influence on the changes in how the individual feels the progress of time on a daily basis is small. On the other hand, the present inventor has considered that the amount of physical exercise and the amount of burden are factors that significantly change every day, and therefore the influence on the changes in how the individual feels the progress of time on a daily basis is large.

More specifically, from the inventor's own actual experience, the present inventor estimates that, in a case where the amount of physical exercise (amount of burden) on the day is large, the progress of time of the user time 412 is faster than the progress of time of the standard time 410. Moreover, on the basis of such estimation, the present inventor has originally come up with the idea that the difference between the amount of exercise of the day and a reference value having a predetermined value (details of the reference value will be described later) is treated as an index of the difference in the progress of time of the user time 412 with respect to the standard time 410 (that is, time difference). Then, on the basis of such an original idea of the present inventor, by using the difference between the amount of exercise of the day and the above reference value, which is an index of the difference in the progress of time of the user time 412 with respect to the standard time 410, the user time 412 can be calculated from the standard time 410. Furthermore, the present inventor estimates the above-mentioned amount of exercise and the like are on the basis of individual biological information (for example, pulse rate and the like).

On the basis of such an original idea of the present inventor, the embodiment of the present disclosure described below has been created. That is, according to the embodiment of the present disclosure, on the basis of the factors of the individual's actions (for example, the amount of exercise and the amount of burden), it is possible to provide the user time 412, which is an individual's time for the individual, created by the individual itself, and to be for the individual. The concept of the embodiment of the present disclosure created by the present inventor will be described below.

### 1.2. Concept

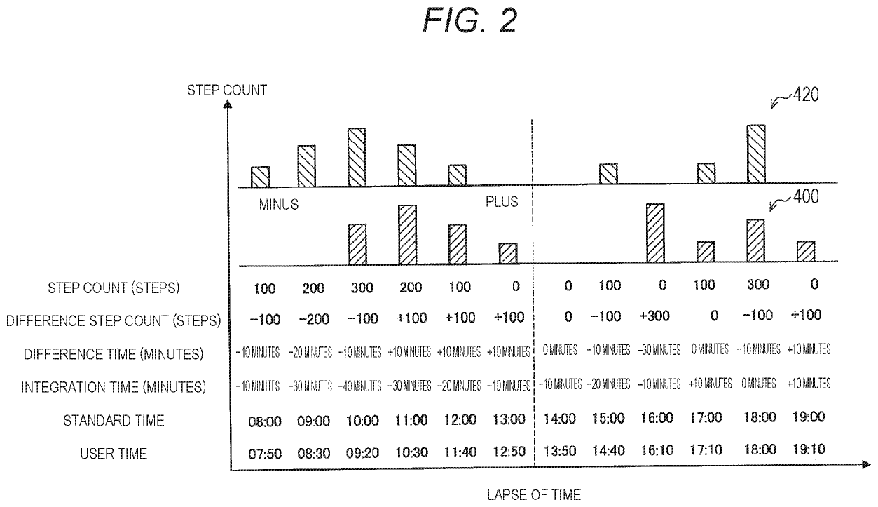

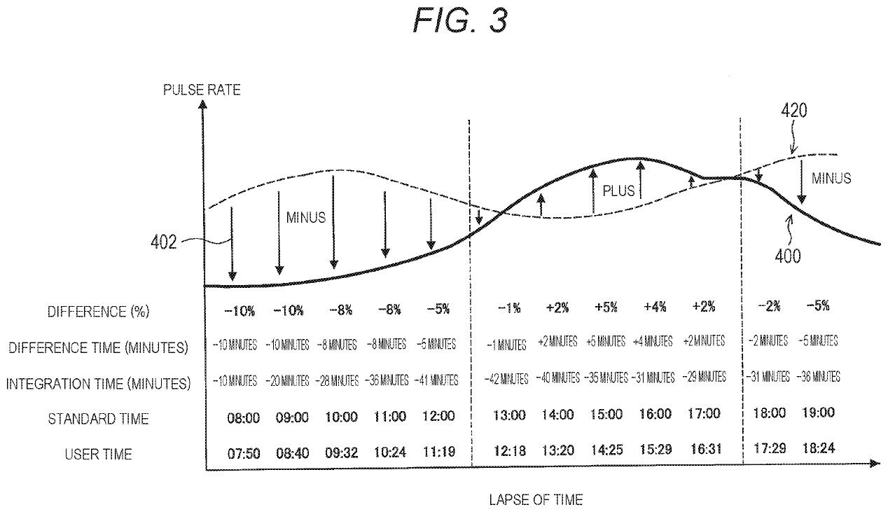

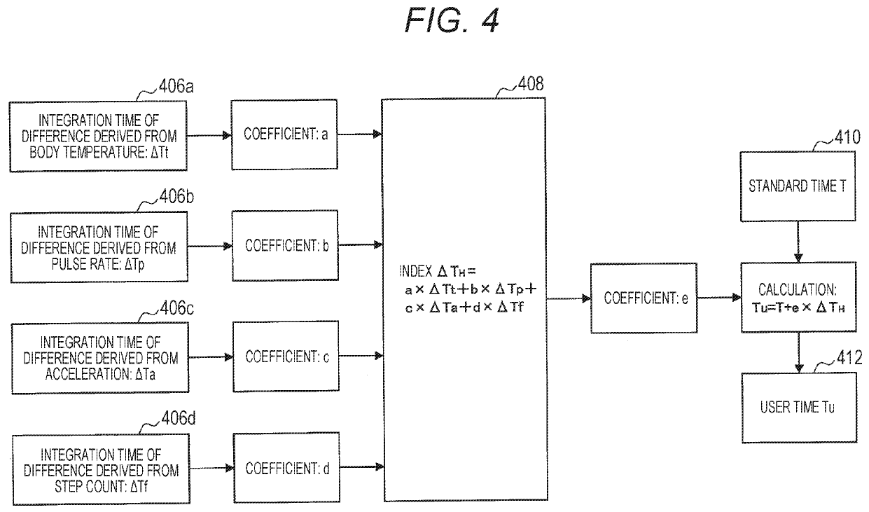

The concept of the embodiment of the present disclosure will be described with reference to FIGS. 1 to 4. FIGS. 1 and 4 are explanatory diagrams for explaining the concept of the embodiment of the present disclosure. FIG. 2 is an explanatory diagram for explaining an example of calculation of the user time according to the embodiment of the present disclosure, and in detail, an example of calculating the user time on the basis of sensing data (temporal change) of the step count of an individual (user) is shown. FIG. 3 is an explanatory diagram for explaining an example of calculation of the user time according to the embodiment of the present disclosure, and in detail, an example of calculating the user time on the basis of sensing data of the pulse rate of the user.

As shown in FIG. 1, in the present embodiment, for example, as various biological information corresponding to the amount of exercise or amount of burden of the individual (user), sensing data (temporal change of first biological information) 400a, 400b, 400c, and 400d are acquired from a body surface temperature sensor 120a, a pulse wave sensor 120b, an acceleration sensor 120c, and a step count sensor 120d. In the present embodiment, the sensing data 400a to 400d (for example, temporal changes in, for example, body temperature, pulse wave, acceleration, step count, and the like) obtained from each of these biological information sensors 120a to 120d are assumed to be related to the amount of exercise and amount of burden of the user. That is, in the present embodiment, it is assumed that the above-mentioned sensing data 400a to 400d are data reflecting the amount of exercise or the amount of burden, which are factors that influence the change in how the user feels the progress of time on a daily basis.

Specifically, in the present embodiment, on the basis of the original estimation that, in a case where the amount of exercise (amount of burden) is large, the progress of time of the user time 412 is faster than the progress of time of the standard time 410, the difference (magnitude relationship) from the reference value is interpreted as described below according to the type of the sensing data 400.

For example, in a case where the sensing data 400a obtained by the body surface temperature sensor 120a such as the user's body temperature is larger than the reference value, it is assumed that the amount of exercise (amount of burden) of the user is large and the progress of time of the user time 412 is interpreted to be faster than the progress of time of the standard time 410. For example, in a case where the sensing data 400b obtained by the pulse wave sensor 120b such as pulse rate or heart rate is larger than the reference value, it is assumed that the amount of physical burden of the user is large and the progress of time of the user time 412 is interpreted to be faster than the progress of time of the standard time 410. Furthermore, for example, in a case where the sensing data 400c obtained by the acceleration sensor 120c such as acceleration is larger than the reference value, it is assumed that the amount of exercise (amount of burden) is large and the progress of time of the user time 412 is interpreted to be faster than the progress of time of the standard time 410. Moreover, for example, in a case where the sensing data 400d obtained by the step count sensor 120d such as the step count is larger than the reference value, it is assumed that the amount of exercise (amount of burden) is large and the progress of time of the user time 412 is interpreted to be faster than the progress of time of the standard time 410.

Moreover, in the present embodiment, in a case where the sensing data 400 obtained by the pulse wave sensor 120b or a brain wave sensor (not shown) indicates that the user is more relaxed with respect to the reference value, it may be assumed that the amount of burden is small and the progress of time of the user time 412 may be interpreted to be slower than the progress of time of the standard time 410. Furthermore, in the present embodiment, in a case where the sensing data 400 obtained by the pulse wave sensor 120b and the like indicates that the user is tense with respect to the reference value, it may be assumed that the amount of burden is large and the progress of time of the user time 412 may be interpreted to be faster than the progress of time of the standard time 410. Moreover, in the present embodiment, in a case where the sensing data 400 obtained by the pulse wave sensor 120b and the like indicates that the user is sleeping for a longer time or the sleep depth is deeper with respect to the reference value, it may be assumed that the amount of burden has decreased and the progress of time of the user time 412 may be interpreted to be slower than the progress of time of the standard time 410.

Table 1 below shows an example of interpretation (assumption) of the progress of time of the user time 412 in various sensing data 400 in the present embodiment. Note that, in the present embodiment, the interpretation is not limited to that shown in Table 1 below.

TABLE 1

| | | | Progress of time |
|---|---|---|---|
| | | Amount of exercise | (relative to |
| Sensing data | Comparison result | (amount of burden) | standard time) |
| Body temperature | Larger than | Large amount of | Faster |
| | reference value | exercise | |
| | (average) (plus | | |
| | difference) | | |
| Pulse rate/heart | Larger than | Large amount of | Faster |
| rate | reference value | burden | |
| | (average) (plus | | |
| | difference) | | |
| Acceleration | Larger than | Large amount of | Faster |
| | reference value | exercise | |
| | (plus difference) | | |
| Step count | Larger than | Large amount of | Faster |
| | reference value | exercise | |
| | (plus difference) | | |

Moreover, in the present embodiment, as shown in FIG. 1, by applying a synthesis algorithm 600 to these sensing data 400a to 400d (in detail, the difference between the sensing data 400 and the reference value), an index 408 related to the user is calculated (see FIG. 4). The index 408 related to this user is an index showing how the user feels the progress of time, and in detail, an index related to the time difference indicating how much the user time 412 is behind or ahead of the standard time 410. Then, in the present embodiment, as shown in FIG. 1, the user time 412 can be calculated by adding the calculated index 408 related to the user to the standard time 410.

Next, the calculation of the index 408 related to the user described above, that is, the details of the synthesis algorithm 600 of the present embodiment will be sequentially described.

First, in the present embodiment, for each sensing data 400, the difference 402 (or difference rate (%)) between the sensing data 400 in a first section and reference data (temporal change in second biological information) 420, which is the same type of sensing data as the sensing data 400, in a second section having the same time as the first section is calculated at predetermined time intervals. Then, in the present embodiment, the calculated difference 402 is multiplied by a predetermined coefficient to convert the difference 402 into a difference time (time conversion), and a plurality of difference times is integrated to calculate the integration time 406 of difference for each sensing data 400 (see FIG. 4). Moreover, in the present embodiment, the integration times 406 of the plurality of sensing data 400 are synthesized by processing on the basis of a predetermined formula to calculate the time difference as the index 408 related to the user.

In the present embodiment, for example, as shown in FIG. 2, as the sensing data 400 of the user, the temporal change in the step count of the user counted at predetermined time intervals (50 minutes in the example of FIG. 2) in the first section (in the example of FIG. 2, the section shown as 8:00 to 19:00 in the standard time 410) is acquired. In other words, in FIG. 2, the sensing data 400 is shown as an example in which the temporal change in the step count of the user is acquired as an example of the temporal change by a group of discretely acquired values. Furthermore, in the present embodiment, as the reference data 420 (reference value), a temporal change in the step count of the user on a day earlier than the day when the sensing data 400 was acquired and having the same time (in the example of FIG. 2, the section shown as 8:00 to 19:00 in the standard time 410) and the same time length (in the example of FIG. 2, it becomes 11 hours in the standard time 410) as the first section is acquired. Moreover, the temporal change in the step count of the user acquired as the reference data 420 is a temporal change in the step count of the user counted at predetermined time intervals (50 minutes in the example of FIG. 2) in the second section similarly to the sensing data 400.

Note that, in the example shown in FIG. 2, the reference data 420 may be a temporal change by a group of smoothed values (mean values) of the step count of the user counted at predetermined time intervals of a plurality of second sections having the same time and the same time length as the first section in a period of the predetermined number of days (for example, about 1 to 3 months) the latest to the day when the sensing data 400 was acquired. Alternatively, the reference data 420 may be reference data set by the user, or may be a temporal change in the step count of another user, and is not particularly limited in the present embodiment.

In more detail, in the present embodiment, the reference data 420 can be selected properly depending on the type of sensing data 400 and what kind of information is desired (for example, the user time 412 of the present time or whether the previous transition of the user time 412 is to be presented to the user, or the like). Moreover, in the present embodiment, the sensing data 400 and the reference data 420 may be subject to processing or the like for removing measurement noise and the like included in the sensing data 400 and the reference data 420, depending on the type of the sensing data 400, what kind of information is desired, and the like.

Then, in the present embodiment, the difference step count is acquired as the difference 402 by subtracting the reference data 420 from the sensing data 400 at predetermined time intervals. For example, in the example of FIG. 2, at 8:00 in the standard time 410, the difference step count is “minus 100 steps” by subtracting “100 steps” of the reference data 420 from “0 steps” of the sensing data 400. Note that, in the present embodiment, the difference 402 may be, for example, the numerical value of the difference itself, or may be converted into a difference rate by performing predetermined statistical processing.

Next, in the present embodiment, the calculated difference 402 is multiplied by a predetermined coefficient to convert it into a difference time. In the example shown in FIG. 2, the difference time is calculated so as to correspond to 10 minutes per 100 steps in the difference 402. Note that, here, since the sensing data 400 is a temporal change in the step count, the difference 402 of positive number is interpreted such that the progress of time of the user time 412 is faster than the progress of time of the standard time 410. Therefore, the difference 402 of positive number is converted into a positive number difference time. On the other hand, since the sensing data 400 is a temporal change in the step count, the difference 402 of negative number is interpreted such that the progress of time of the user time 412 is slower than the progress of time of the standard time 410. Therefore, the difference 402 of negative number is converted into a negative number difference time (see Table 1). More specifically, in the example of FIG. 2, in a case where the difference 402 is minus 100 steps, the difference time is converted into minus 10 minutes.

Moreover, in the present embodiment, the difference integration time 406 is acquired by integrating a plurality of integration times obtained from a predetermined start time set by the user or the like (for example, set by the time indicated in the standard time 410. In the example of FIG. 2, “8:00” in the standard time 410) to a predetermined end time set by the user or the like (for example, set by the time indicated in the standard time 410. In the example of FIG. 2, “19:00” in the standard time 410). For example, in the example of FIG. 2, the integration time 406 is “plus 10 minutes” at 19:00 in the standard time.

Then, in the present embodiment, the resulting integration times 406 of the plurality of different types of sensing data 400 are synthesized by processing on the basis of a predetermined formula so that the synthesized integration time 406 is calculated as the index 408 related to the user (time difference). Then, in the present embodiment, the user time 412 can be calculated by adding the synthesized integration time 406, which is the index 408 related to the user, to the standard time 410.

Note that, in the example shown in FIG. 2, a case is shown in which the synthesis with the integration time 406 related to the other type of sensing data 400 is omitted, and the user time 412 is directly calculated only on the basis of the integration time 406 related to the sensing data 400, which is the temporal change in the step count of the user. In other words, in the example shown in FIG. 2, the integration time 406 related to the sensing data 400, which is the temporal change in the step count of the user, is treated as the index 408 related to the user. For example, in the example of FIG. 2, since the integration time 406 is “plus 10 minutes” at 19:00 in the standard time, it is directly added to calculate “19:10” as the user time 412. Note that the details of the above synthesis will be described later.

In the present embodiment, as described above, the user time 412 may be calculated on the basis of one piece of sensing data 400, but it is preferable to perform synthesis to calculate the user time 412 on the basis of a plurality of different types of sensing data 400. This is because, in the present embodiment, by using a plurality of different types of sensing data 400, it is considered that the possibility of obtaining a highly accurate user time 412 that is closer to the actual feeling of the user is increased. Moreover, by doing so, even in a case where the reliability of one or more sensing data 400 is low (deterioration of measurement accuracy, measurement state, or the like), the user time 412 can be calculated on the basis of the remaining other highly reliable sensing data 400, and it is possible to provide the user time 412 continuously. Note that, in the following description, the highly accurate user time 412 means the user time 412 that is close to the user's actual feeling or that the user time 412 is calculated by faithfully reflecting the physical condition (amount of exercise, amount of load) and the like of the user.

Furthermore, as another specific example, as shown in FIG. 3, as the sensing data 400 of the user, the temporal change in the pulse rate of the user obtained at predetermined time intervals in the first section (in the example of FIG. 3, the section shown as 8:00 to 19:00 in the standard time 410) is acquired. In other words, in FIG. 3, the sensing data 400 is shown as an example in which the temporal change in the pulse rate of the user is acquired as an example of the temporal change in a continuously sensed value. Moreover, in the present embodiment, for example, as the reference data 420, a temporal change in the pulse rate of the user obtained at predetermined time intervals in the second section on a day earlier than the day when the sensing data 400 was acquired and having the same time (in the example of FIG. 3, the section shown as 8:00 to 19:00 in the standard time 410) and the same time length (in the example of FIG. 3, it becomes 11 hours in the standard time 410) as the first section is acquired. Note that, also in the example shown in FIG. 3, the reference data 420 may be a temporal change in smoothed values (mean values) of the pulse rate of the user obtained at predetermined time intervals of a plurality of second sections having the same time and the same time length as the first section in a period of the predetermined number of days (for example, about 3 to 5 days) the latest to the day when the sensing data 400 was acquired, and is not particularly limited.

Then, in the present embodiment, a difference pulse rate (%) can be obtained as the difference 402 by subtracting the reference data 420 from the sensing data 400 and performing normalization with the reference data or the like of the corresponding time at predetermined time intervals (50 minutes in the example of FIG. 3).

Next, in the present embodiment, the calculated difference 402 is multiplied by a predetermined coefficient to convert it into a difference time (time conversion). In the example shown in FIG. 3, the difference time is calculated so as to correspond to 10 minutes per 10% in the difference 402. Note that, here, since the sensing data 400 is a temporal change in the pulse rate, the difference 402 of positive number is interpreted such that the progress of time of the user time 412 is faster than the progress of time of the standard time 410. Therefore, the difference 402 of positive number is converted into a positive number difference time. On the other hand, since the sensing data 400 is a temporal change in the pulse rate, the difference 402 of negative number is interpreted such that the progress of time of the user time 412 is slower than the progress of time of the standard time 410. Therefore, the difference 402 of negative number is converted into a negative number difference time (see Table 1). Specifically, in the example of FIG. 3, in a case where the difference 402 is minus 10%, the difference time is converted into minus 10 minutes.

Moreover, in the present embodiment, the difference integration time 406 is acquired by integrating a plurality of difference times obtained from a predetermined start time set by the user or the like (in the example of FIG. 3, “8:00” in the standard time 410) to a predetermined end time set by the user or the like (in the example of FIG. 3, “19:00” in the standard time 410). Specifically, in the example of FIG. 3, the integration time 406 is “minus 36 minutes” at 19:00 in the standard time.

Then, in the example shown in FIG. 3, a case is shown in which the synthesis with the integration time 406 related to the other type of sensing data 400 is omitted, and the user time 412 is directly calculated only on the basis of the integration time 406 related to the sensing data 400, which is the temporal change in the pulse rate of the user. In other words, in the example shown in FIG. 3, the integration time 406 related to the sensing data 400, which is the temporal change in the pulse rate of the user, is treated as the index 408 related to the user. Specifically, in the example of FIG. 3, since the integration time 406 is “minus 36 minutes” at 19:00 in the standard time, it is directly added to calculate “18:24” as the user time 412.

Moreover, as described above, in the present embodiment, it is preferable that the resulting integration times 406 of the plurality of different types of sensing data 400 be synthesized by processing on the basis of a predetermined formula to calculate the index 408 related to the user. For example, in the present embodiment, as shown in FIG. 4, integration times 406a to 406d related to (derived from) each sensing data (in the example of FIG. 4, each of the integration times 406a to 406d related to each sensing data is indicated at ΔTt, ΔTp, ΔTa, ΔTf) are multiplied by the coefficients (weighting) a to d predetermined on the basis of the characteristics of each sensing data 400. Moreover, in the present embodiment, by adding the multiplied integration times 406a to 406d, the added integration times 406a to 406d are calculated as the index (time difference) 408 related to the user (in the example of FIG. 4, the index 408 related to the user is indicated at ΔTH). That is, the index 408 related to the user can be calculated by using the following formula (1).

[Math. 1]

ΔTH=a×ΔTₜ+b×ΔTₚ+c×ΔTₐ+d×ΔT_{f} Formula (1)

Moreover, in the present embodiment, the index 408 related to the user calculated as described above is multiplied by the predetermined coefficient e (for example, a coefficient e determined according to the attributes of the user), and the result is added to the standard time 410 (in the example of FIG. 4, the standard time is indicated at T) so that the user time 412 (in the example of FIG. 4, the user time 412 is indicated at Tu) can be calculated. That is, the user time 412 can be calculated using the following formula (2).

[Math. 2]

Tᵤ=e×ΔT_{H}+T Formula (2)

Note that, in the present embodiment, for example, the above-mentioned coefficients a to e can be set as described below. The coefficients a to e can be set, for example, by using the difference (change amount) from the sensing data of the user obtained in the latest (for example, the previous day), or a statistical index such as variance obtained by statistically processing the plurality of sensing data obtained in the latest (for example, the immediately preceding 3 to 5 days). Furthermore, in the present embodiment, the coefficients a to e may be values calculated on the basis of the sensing data of a plurality of users including other users. Moreover, in the present embodiment, they may be set according to the attribute information of the user (age, gender, and the like) and the environmental information around the user (temperature, season, and the like). Then, as described above, the coefficients a to e preferably set for calculating the user time 412 for each user may be associated with each user or the attribute information of each user and may be stored and used in the storage unit 308 of the server 30.

In the embodiment of the present disclosure, the user time 412 can be calculated on the basis of the concept described above. Note that the examples shown in FIGS. 1 to 4 are shown as an example of the present embodiment, and the embodiment of the present disclosure is not limited to the examples shown in FIGS. 1 to 4. Next, the information processing system according to the embodiment of the present disclosure, which calculates the user time 412 using the concept described above, will be described.

### 2. Embodiment of the Present Disclosure

### 2.1. Configuration of the Information Processing System 1 According to the Embodiment of the Present Disclosure

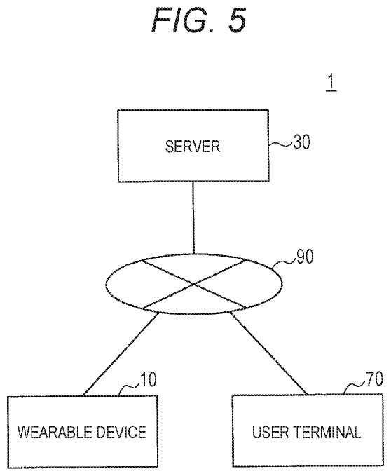

A configuration of the information processing system 1 according to the embodiment of the present disclosure is described with reference to FIG. 5. FIG. 5 is an explanatory diagram for explaining an example of a configuration of the information processing system 1 according to the present embodiment.

As shown in FIG. 5, the information processing system 1 according to the present embodiment includes a wearable device (wearable terminal) 10, a server 30, and a user terminal 70, which are communicably connected to each other via a network 90. In detail, the wearable device 10, the server 30, and the user terminal 70 are connected to the network 90 via a base station (for example, a mobile phone base station, a wireless local area network (LAN) access point, and the like), which is not shown. Note that as the communication scheme used in the network 90, any scheme can be applied regardless of whether it is wired or wireless (for example, WiFi (registered trademark), Bluetooth (registered trademark), and the like), but it is desirable to use a communication scheme that can maintain stable operation.

(Wearable Device 10)

The wearable device 10 can be a device that can be attached to a part of the body of the user (earlobe, neck, arm, wrist, ankle, and the like) or an implant device (implant terminal) inserted into the body of the user. More specifically, the wearable device 10 can be various types of a wearable device such as a head mounted display (HMD) type, an eyeglass type, an ear device type, an anklet type, a bracelet (wristband) type, a collar type, an eyewear type, a pad type, a batch type, and a clothing type.

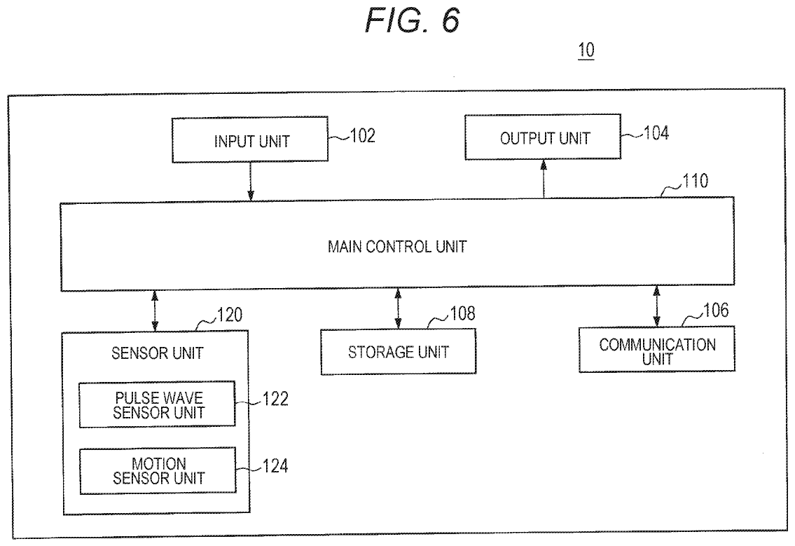

Moreover, the wearable device 10 has, for example, a sensor unit (biological information sensor) 120 incorporating sensors such as a pulse wave sensor unit 122 that detects the pulse of the user (see FIG. 6). In the present embodiment, the above-mentioned user time 412 can be calculated on the basis of the sensing data 400 acquired by such a sensor unit 120. Furthermore, in the present embodiment, the step count, the sleep state (sleep depth, sleep time), and the like of the user may be estimated on the basis of the sensing data acquired by the sensor unit 120 of the wearable device 10, and the estimation result may be used as the sensing data 400. Note that in the present embodiment, the sensor unit 120 may be provided as a body separate from the wearable device 10. Furthermore, in the following description, the wearable device 10 will be described as being a bracelet (wristband) type wearable device. Moreover, the detailed configuration of the wearable device 10 will be described later.

(Server 30)

The server 30 includes, for example, a computer or the like. The server 30 is owned by, for example, a service provider who provides services according to the present embodiment, and can provide (present) services (for example, provision of the user time 412) to each user. Specifically, the server 30 calculates the user time 412 on the basis of the sensing data 400 from each wearable device 10, and provides the calculated user time 412 to the user via the wearable device 10 or the user terminal 70. Note that the detailed configuration of the server 30 will be described later.

(User Terminal 70)

The user terminal 70 is a terminal used by the user or installed in the vicinity of the user to output the information obtained by the server 30 (for example, the user time 412) to the user. Furthermore, the user terminal 70 may receive the information input from the user and transmit the received information to the server 30. For example, the user terminal 70 can be a mobile terminal such as a tablet personal computer (PC), a smartphone, a mobile phone, a laptop PC, a notebook PC, or a wearable device such as an HMD. Moreover, in detail, the user terminal 70 may include a display unit (not shown) that performs a display to the user, an operation unit (not shown) that accepts operations from the user, a speaker (not shown) that performs sound output to the user, and the like.

Note that, in FIG. 1, the information processing system 1 according to the present embodiment is shown as including one wearable device 10 and one user terminal 70, but the present embodiment is not limited to this. For example, the information processing system 1 according to the present embodiment may include a plurality of wearable devices 10 and a plurality of user terminals 70. Moreover, the information processing system 1 according to the present embodiment may include, for example, another communication apparatus such as a relay apparatus for transmitting information from the wearable device 10 to the server 30. Furthermore, the information processing system 1 according to the present embodiment may not include the wearable device 10. In such a case, for example, the user terminal 70 functions like the wearable device 10 and the sensing data acquired by the user terminal 70 is output to the server 30. Moreover, the information processing system 1 according to the present embodiment may not include the user terminal 70. In such a case, for example, the wearable device 10 functions like the user terminal 70 and the information acquired from the server 30 is output to the wearable device 10.

### 2.2. Configuration of a Wearable Device 10 According to the Embodiment of the Present Disclosure

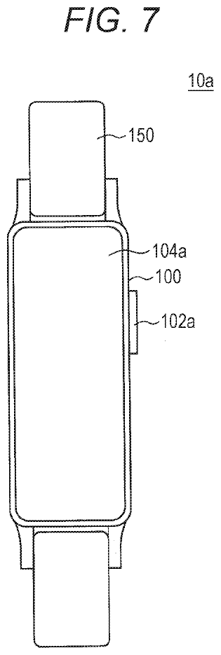

The configuration of the information processing system 1 according to the embodiment of the present disclosure has been described above. Next, the configuration of the wearable device 10 according to the embodiment of the present disclosure will be described with reference to FIGS. 6 and 7. FIG. 6 is a block diagram showing an example of the configuration of the wearable device 10 according to the present embodiment, and FIG. 7 is an explanatory diagram for explaining an example of the appearance of the wearable device 10 according to the present embodiment.

As shown in FIG. 6, the wearable device 10 mainly includes an input unit 102, an output unit (presentation unit) 104, a communication unit 106, a storage unit 108, a main control unit 110, and a sensor unit 120. The details of each functional unit of the wearable device 10 will be described below.

(Input Unit 102)

The input unit 102 receives input of data and commands from the user to the wearable device 10. More specifically, the input unit 102 is realized by a touch panel, a button, a switch, a dial, a microphone, or the like. Furthermore, in the present embodiment, the wearable device 10 may not acquire direct input from the user, but may detect the user's motion with a motion sensor unit 124 described later and acquire input information on the basis of the sensing data 400 related to the detected user's motion.

(Output Unit 104)

The output unit 104 is a functional unit for presenting information to the user, and outputs various information to the user, for example, by image, sound, color, light, vibration, or the like. More specifically, the output unit 104 can present the user time 412, the index 408 related to the user, and the like to the user by displaying the user time 412 provided from the server 30 described later on the screen. The output unit 104 is realized by a display, a speaker, earphones, a light emitting element (for example, a light emitting diode (LED)), a vibration module, or the like. Note that a part of the function of the output unit 104 may be provided by the user terminal 70.

(Communication Unit 106)

The communication unit 106 is provided in the wearable device 10 and can transmit/receive information to/from an external apparatus such as the server 30. In other words, the communication unit 106 can be said to be a communication interface having a function of transmitting and receiving data. The communication unit 106 is realized by, for example, a communication device such as a communication antenna, a transmission/reception circuit, and a port.

(Storage Unit 108)

The storage unit 108 is provided in the wearable device 10 and stores programs, information, and the like for the main control unit 110, which will be described later, to execute various processing, and information obtained by the processing. The storage unit 108 is realized by, for example, a nonvolatile memory such as a flash memory.

(Main Control Unit 110)

The main control unit 110 is provided in the wearable device 10 and can control each functional unit of the wearable device 10. For example, the main control unit 110 acquires the sensing data 400 from the sensor unit 120 described later, converts it into a predetermined format that can be transmitted, and transmits the sensing data 400 in the predetermined format to the server 30 described later via the communication unit 106. Moreover, the main control unit 110 may incorporate a clock mechanism (not shown) for grasping an accurate time, and present the standard time 410 obtained from the clock mechanism to the user via the output unit 104 described above. The main control unit 110 is realized, for example, by hardware such as a central processing unit (CPU), a read only memory (ROM), a random access memory (RAM), and the like. Note that a part of the function of the main control unit 110 may be provided by the server 30 described later.

(Sensor Unit 120)

The sensor unit 120 is provided in the wearable device 10 mounted on the user's body, and includes a pulse wave sensor unit (beat sensor) 122 that detects the pulse of a target user, a motion sensor unit 124 that detects the movement of the user's body, and the like The details of the various sensors included in the sensor unit 120 will be described below.

—Pulse Wave Sensor Unit 122—

The pulse wave sensor unit 122 is a biosensor that is attached to a part of the body such as the skin of the user (for example, both arms, wrists, ankles, and the like) in order to detect the pulse of the user and detects the pulse wave of the user. Here, the pulse wave means a waveform due to the beat of arteries that appears on the surface of the body or the like when the muscles of the heart contract at a constant rhythm (beat; note that the number of times of beat in the heart for a unit time is called the heart rate), the blood is sent to the whole body through the arteries and changes the pressure on the inner wall of the arteries. For example, in order to acquire a pulse wave, the pulse wave sensor unit 122 irradiates a blood vessel in a user's measurement site such as a hand, arm, or leg with light, and detects the light scattered in a substance moving in the user's blood vessel or a stationary living tissue. Since the irradiation light is absorbed by the red blood cells in the blood vessel, the amount of light absorbed is proportional to the amount of blood flowing in the blood vessel in the measurement site. Therefore, the pulse wave sensor unit 122 can know the change in the amount of flowing blood by detecting the intensity of the scattered light. Moreover, the beat waveform (pulse wave) can be detected from the change in blood flow rate, and the pulse can be detected from the change in the waveform per predetermined time. Note that such a method is called a photoplethysmography (PPG) method.

In detail, the pulse wave sensor unit 122 incorporates, for example, a small laser or LED (not shown) capable of emitting coherent light, and emits light having a predetermined wavelength such as around 850 nm. Note that, in the present embodiment, the wavelength of the light emitted by the pulse wave sensor unit 122 can be appropriately selected. Moreover, the pulse wave sensor unit 122 incorporates, for example, a photodiode (photo detector (PD)) and acquires a pulse wave by converting the detected light intensity into an electric signal. Note that the pulse wave sensor unit 122 may incorporate a charge coupled devices (CCD) sensor, a complementary metal oxide semiconductor (CMOS) sensor, or the like instead of the PD. Furthermore, the pulse wave sensor unit 122 may include an optical system mechanism such as a lens or a filter in order to detect light from the measurement site of the user. Then, the pulse wave sensor unit 122 can detect a pulse wave (sensing data 400) as a temporal change having a plurality of peaks, and by counting the plurality of peaks appearing in the pulse wave per predetermined time, the pulse rate of the user can be detected.

Moreover, in the present embodiment, by statistically performing various processing on the pulse wave thus obtained (for example, the temporal change in the peak interval time in the pulse wave is acquired and the acquisition result is analyzed), the user's sleep time, sleep depth, degree of relaxation, degree of tension, and the like may be calculated.

Furthermore, the present embodiment is not limited to acquiring the pulse wave by using the PPG method described above, but the pulse wave may be acquired by another method. For example, in the present embodiment, the pulse wave sensor unit 122 may detect the pulse wave by using a laser Doppler blood flow measurement method. The laser Doppler blood flow measurement method is a method of measuring blood flow by utilizing the phenomenon described below. In detail, when a laser beam is emitted to the measurement site of the user, scattered light accompanied by a Doppler shift is generated due to the movement of scattering substances (mainly red blood cells) existing in the blood vessel of the user. Then, the scattered light accompanied by the Doppler shift interferes with the scattered light by the non-moving tissues existing in the measurement site of the user, and a beat-like intensity change is observed. Therefore, the laser Doppler blood flow measurement method can detect a pulse wave by analyzing the intensity and frequency of a beat signal.

Note that, in the present embodiment, instead of the pulse wave sensor unit 122, an electrocardiogram (ECG) sensor unit (not shown) that detects the electrocardiogram of the user via an electrode (not shown) attached to the user's body may be provided. In this case, the user's heart rate can be detected from the detected electrocardiogram.

Furthermore, in the present embodiment, the sensor unit 120 may include various other biological information sensors (not shown) in place of the pulse wave sensor unit 122 or together with the pulse wave sensor unit 122. For example, the various biological information sensors can include one or a plurality of sensors that are directly or indirectly attached to a part of the body of the target user to measure brain waves, respiration, myoelectric potential, skin temperature, sweating, blood pressure, blood oxygen concentration, and the like of the target user.

—Motion Sensor Unit 124—

Furthermore, the sensor unit 120 may include a motion sensor unit 124 for detecting the movement of the user's body. The motion sensor unit 124 detects the step count of the user or the like on the basis of the amount of exercise of the user or the movement distance of the user, for example, by acquiring the sensing data 400 indicating the change in acceleration generated by the movement of the user. Specifically, the motion sensor unit 124 includes an acceleration sensor, a gyro sensor, a geomagnetic sensor, and the like (not shown).

Moreover, the sensor unit 120 may include a positioning sensor (position sensor) (not shown) instead of the motion sensor unit 124 or together with the motion sensor unit 124. The positioning sensor is a sensor that detects the position of the user wearing the wearable device 10, and can be specifically a global navigation satellite system (GNSS) receiver or the like. In this case, the positioning sensor can generate the sensing data 400 indicating the latitude and longitude of the target user's current location on the basis of a signal from a GNSS satellite. Furthermore, in the present embodiment, for example, it is possible to detect the relative positional relationship of the user from radio frequency identification (RFID), a Wi-Fi access point, radio base station information, and the like, and it is also possible to use such a communication apparatus as the positioning sensor.

As described above, in the present embodiment, the sensor unit 120 can include various biological information sensors and the like. Moreover, the sensor unit 120 may cooperate with the clock mechanism (not shown) included in the main control unit 110 described above, and may associate the acquired sensing data 400 with the standard time 410 at which the sensing data 400 has been acquired. Furthermore, the various sensors may not be provided in the sensor unit 120 of the wearable device 10, and may, for example, be provided as a body separate from the wearable device 10.

Moreover, as described above, the wearable device 10 can adopt various types of a wearable device such as an HMD type, an ear device type, an anklet type, a bracelet type, a collar type, an eyewear type, a pad type, a batch type, and a clothing type. For example, a wearable device 10a shown in FIG. 7 is a bracelet (wristband) type wearable device. The wearable device 10a includes a main body 100, a button 102a provided on the side surface of the main body 100 for the user to operate the wearable device 10a (for example, the number of the buttons 102a is not limited to one, but may be plural), and a display unit 104a provided on the surface of the main body 100 and includes for example an organic electro luminescence (EL) display or the like. Moreover, the wearable device 10a has a wristband 150 for attaching and fixing the main body 100 to the user's arm. Furthermore, the main body 100 may incorporate a universal serial bus (USB) port (not shown) as an interface for connecting an external apparatus, a battery such as a Li-ion battery (not shown), and the like.

Note that the wearable device 10 shown in FIGS. 6 and 7 is an example of the present embodiment. That is, in the present embodiment, the wearable device 10 is not limited to the examples shown in FIGS. 6 and 7.

### 2.3. Configuration of the Server 30 According to the Embodiment of the Present Disclosure

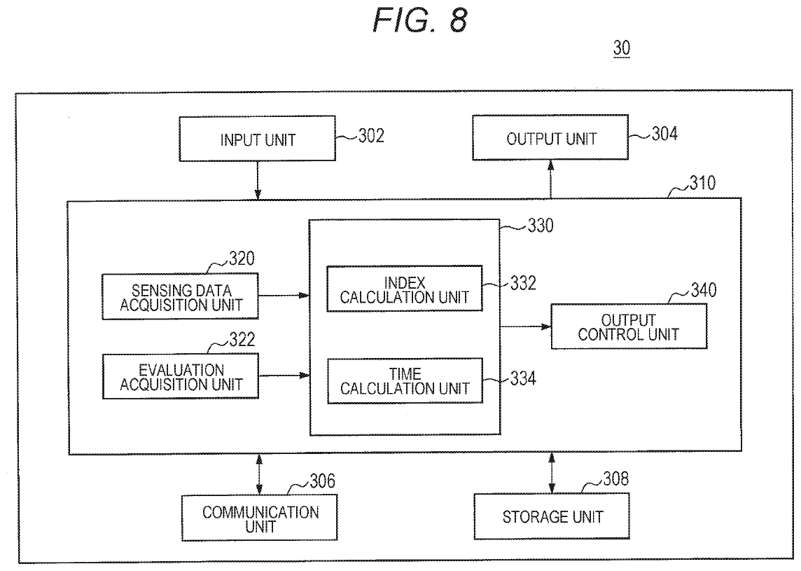

The configuration of the wearable device 10 according to the embodiment of the present disclosure has been described above. Next, the configuration of the server 30 according to the embodiment of the present disclosure will be described with reference to FIG. 8. FIG. 8 is a block diagram showing an example of the configuration of the server 30 according to the present embodiment.

As described above, the server 30 includes, for example, a computer or the like. As shown in FIG. 8, the server 30 mainly includes an input unit 302, an output unit 304, a communication unit 306, a storage unit 308, and a main control unit 310. The details of each functional unit of the server 30 will be described below.

(Input Unit 302)

The input unit 302 accepts input of data and commands to the server 30. More specifically, the input unit 302 is realized by, for example, a touch panel, a keyboard, or the like.

(Output Unit 304)

The output unit 304 includes, for example, a display, a speaker, a video output terminal, a sound output terminal, and the like, and outputs various information by an image, a sound, or the like.

(Communication Unit 306)

The communication unit 306 is provided in the server 30 and can transmit and receive information to and from an external apparatus such as the wearable device 10 and the user terminal 70. The communication unit 306 is realized by, for example, a communication device such as a communication antenna, a transmission/reception circuit, and a port.

(Storage Unit 308)

The storage unit 308 is provided in the server 30 and stores programs, information, and the like for the main control unit 310, which will be described later, to execute various processing, and information obtained by the processing. The storage unit 308 is realized by, for example, a magnetic recording medium such as a hard disk (HD), a nonvolatile memory such as a flash memory, and the like.

(Main Control Unit 310)

The main control unit 310 is provided in the server 30 and can control each block of the server 30 and calculate the user time 412 on the basis of the acquired sensing data 400. The main control unit 310 is realized, for example, by hardware such as a CPU, ROM, and RAM. Furthermore, the main control unit 310 can also function as a sensing data acquisition unit (information acquisition unit) 320, an evaluation acquisition unit 322, a processing unit 330, and an output control unit 340. The details of these functions of the main control unit 310 according to the present embodiment will be described below. Note that the main control unit 310 may execute a part of the function of the main control unit 110 of the wearable device 10, or a part of the function of the main control unit 310 may be executed by the main control unit 110 of the wearable device 10.

—Sensing Data Acquisition Unit 320—

The sensing data acquisition unit 320 acquires a plurality of sensing data (temporal changes) 400 of one or different types output from one or a plurality of wearable devices 10, and outputs the acquired sensing data 400 to the processing unit 330 described later. Moreover, the sensing data acquisition unit 320 may cooperate with the sensor unit 120 of the wearable device 10 in order to suppress an increase in the power consumption of the sensor unit 120 or improve the accuracy of the sensing data 400 to change the timing of acquisition (time intervals) of the sensing data 400 as appropriate.

—Evaluation Acquisition Unit 322—

The evaluation acquisition unit 322 acquires the evaluation and the like of the user time 412 and the index 408 related to the user by the user, and outputs the acquired evaluation and the like to the processing unit 330. For example, the processing unit 330 can change the synthesis algorithm 600 of the user time 412 by referring to the evaluation and the like, and correct the calculated user time 412 to a time closer to the actual feeling of the user.

—Processing Unit 330—

The processing unit 330 processes the sensing data 400 output from the sensing data acquisition unit 320 described above, and calculates the index 408 related to the user and the user time 412. In detail, the processing unit 330 functions as an index calculation unit (calculation unit) 332 and a time calculation unit 334 in order to realize these functions described above. The details of these functions of the processing unit 330 according to the present embodiment will be described below.

The index calculation unit 332 calculates the difference 402 between the sensing data 400 in the first section and the reference data 420 in the second section at the same time as the first section at predetermined time intervals. Moreover, the index calculation unit 332 converts the plurality of calculated differences 402 into time and integrates them to calculate the integration time 406. Furthermore, the index calculation unit 332 calculates the index 408 related to the user related to the time difference from the standard time 410 on the basis of the integration time 406 described above. In detail, the index calculation unit 332 weights each integration time 406 (for example, multiplies a predetermined coefficient) according to the type of sensing data 400, then adds each integration time 406 of a different type, and calculate the added integrated time as the index (time difference) 408 related to the user. At this time, the index calculation unit 332 may select the sensing data 400 to be used when calculating the index 408 related to the user on the basis of the reliability of each sensing data 400. Moreover, the index calculation unit 332 may appropriately change the reference data 420 used for the calculation and may appropriate change the weighting (coefficient for multiplication) for calculation on the basis of the evaluation acquired by the evaluation acquisition unit 322 described above, the attribute information of the user, the schedule of the user, and the like. Furthermore, the index calculation unit 332 may appropriately change the calculation timing (time interval) in order to suppress an increase in the power consumption of the sensor unit 120 or to improve the accuracy of the sensing data 400.

The time calculation unit 334 calculates the user time 412 by adding the index (time difference) 408 related to the user calculated by the index calculation unit 332 to the standard time 410.

—Output Control Unit 340—

The output control unit 340 causes the communication unit 306 described above to transmit the result obtained by the processing unit 330 described above (for example, the index 408 related to the user and the user time 412) to the wearable device 10 or the user terminal 70.

Note that the server 30 shown in FIG. 8 is an example of the present embodiment. That is, in the present embodiment, the server 30 is not limited to the example shown in FIG. 8.

### 2.4. Information Processing Method According to the Embodiment of the Present Disclosure

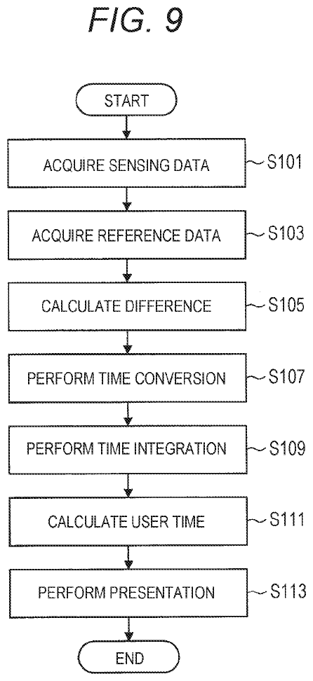

The details of the information processing system 1 according to the embodiment of the present disclosure and each apparatus included in the information processing system 1 have been described above. Next, the information processing method according to the present embodiment will be described with reference to FIG. 9. FIG. 9 is a flowchart showing an example of the information processing method according to the present embodiment.

As shown in FIG. 9, the information processing method according to the present embodiment includes a plurality of steps from step S101 to step S113. The details of each step included in the information processing method according to the present embodiment will be described below.

First, before starting the information processing according to the present embodiment, the wearable device 10, the server 30, or the user terminal 70 receives the user's age, gender, height, weight, and holidays (information related to the user's lifestyle in a week), commuting, school hours (information related to the user's weekday lifestyle) as attribute information from the user, and other information related to the user's specific periodic activities. For example, the user can input the user's own attribute information by answering to a question window (for example, “What is your gender? 1: Male, 2: Female”) displayed on the display unit (not shown) of the user terminal 70 such as a smartphone. Note that, in the present embodiment, the input of attribute information is not limited to being performed before the initial information processing, but may be performed in the middle of continuous information processing, and is particularly limited. Moreover, the wearable device 10, the server 30, or the user terminal 70 may acquire information such as the ambient temperature of the user on the day by using the input from the user, the position information of the user, and the like. Then, the attribute information and the like accepted in this way will be referred to when weighting performed when calculating the user time 412, selecting the reference data 420, and the like. Furthermore, in the present embodiment, input such as the schedule of the day of the user may be accepted together with the attribute information. In the present embodiment, for example, actions such as exercise and drinking are likely to influence how the user feels the progress of time. Therefore, it is preferable to accept the input of the schedule of running, trekking, participating in a drinking party, and the like. Then, in the present embodiment, the accepted schedule information may be referred to when weighting performed when calculating the user time 412, selecting the reference data 420, and the like, similarly to the above attribute information. Moreover, in the present embodiment, the server 30 may store the schedule information in association with the corresponding sensing data 400, the index 408 related to the user, and the information related to the tendency of the user time 412. By doing so, it becomes possible to analyze the influence of the content of the user's action on the user's body and the like, which is reflected in the user time 412 and the like, at a later date.

(Step S101)

The server 30 acquires one or a plurality of different types of sensing data 400 from the wearable device 10.

Note that, in the present embodiment, it is preferable to perform the following processing in order to ensure the quality of the sensing data 400. In detail, regarding the sensing data 400 derived from a pulse wave or the like, the measurement state changes depending on the wearing state of the wearable device 10 including the sensor unit 120 and the influence of the user's physical movement. Therefore, since the sensing data 400 such as the pulse rate is not always acquired in a good measurement state, it is preferable that the sensing data is selected as the sensing data 400 for calculation of the user time 412 after the following processing is performed on the acquired sensing data 400.

Specifically, in the present embodiment, for example, the sensing data is used as the sensing data 400 for calculation of the user time 412 after performing processing in which a threshold value is set in advance for the amplitude of the pulse wave waveform and a waveform portion having an amplitude lower than the threshold value or a waveform portion having a high amplitude is removed. Furthermore, for example, it is determined whether the pulse wave waveform has a waveform far from a noise waveform existing in the vicinity in time, and after performing processing of removing a waveform portion similar to the noise waveform, the sensing data is used as the sensing data 400 for calculation of the user time 412.

Furthermore, since the pulse wave has the property that similar waveforms are detected periodically, it is determined whether the detected pulse wave waveform is located in a time frame that can be estimated from the pulse wave waveform detected immediately before. Moreover, in a case where it is not in the time frame, a dummy pulse wave waveform is arranged in the time frame, and the time frame in which a waveform to be detected next will exist is estimated. In the present embodiment, by repeating such estimation and determination, the reliability of the acquired pulse wave is determined, and on the basis of the determination, it is determined whether or not the pulse wave is selected as the sensing data 400 for calculation of the user time 412. Furthermore, in the present embodiment, for example, the reliability of the pulse wave may be determined by using the sensing data 400 by the motion sensor unit 124.

Note that, in the present embodiment, in a case where the sensing data 400 such as the pulse rate is not selected as the sensing data 400 for calculation of the user time 412 according to the reliability determination result or the like, only another type of sensing data 400 may be used to calculate the user time 412. Alternatively, in the present embodiment, in a case where the another type of sensing data 400 is not acquired or selected, the standard time 410 may be temporarily used as the user time 412. Moreover, in the present embodiment, when presenting the user time 412 to the user, it is preferable to present to the user what type of sensing data 400 has been used to calculate the user time 412.

Furthermore, in the present embodiment, with respect to the sensing data 400 derived from acceleration such as acceleration and step count, it is assumed that these sensing data 400 have high reliability, and the above-mentioned processing may not be performed.

(Step S103)

The server 30 selects the reference data 420 to be compared with the sensing data 400. In the present embodiment, the reference data 420 can be the same type of sensing data as the above-mentioned sensing data acquired from the wearable device 10 worn by the user. Moreover, the reference data 420 can be sensing data acquired at predetermined time intervals in the second section on a day earlier than the day when the sensing data 400 was acquired and having the same time and the same time length as the first section.

More specifically, the reference data 420 may be a temporal change of smoothed values (mean value) acquired at predetermined time intervals of a plurality of second sections in a period of a predetermined number of days satisfying predetermined conditions and the latest to the day when the sensing data 400 was acquired (e.g., the last 3 to 5 days, the last 3 to 5 days of the last weekday, the last 7 days of the last week, the last 4 days of the same day of the week in the last month, the last 4 days of the last month when the user's schedule is the same, and the like) and having the same time and the same time length as the first section described above. For example, when the sensing data 400 is acquired on a weekday, as the reference data 420, data obtained by smoothing a plurality of sensing data for three days of the latest weekday can be used. For example, when the sensing data 400 is acquired on Wednesday, as the reference data 420, data obtained by smoothing a plurality of sensing data for the latest three Wednesdays can be used. Moreover, for example, when the sensing data 400 is the sensing data acquired on the day when the user's schedule includes running, as the reference data 420, data obtained by smoothing a plurality of sensing data of three days when the latest user's schedule includes running can be used.

Alternatively, in the present embodiment, the reference data 420 may be sensing data acquired at predetermined time intervals of the second section on a past day set by the user (for example, the previous day, the latest past weekday, the latest past same day of the week, the same month and date of last year, and the like) having the same time and the same time length as the first section described above. Furthermore, in the present embodiment, the reference data 420 may be sensing data acquired from the wearable device 10 worn by another user or may be a model of sensing data previously stored in the storage unit 308 of the server 30 (default data).

Moreover, in the present embodiment, the reference data 420 can be appropriately changed depending on the attribute information of the user, what kind of information is desired, and the like. For example, when the user is a male, the sensing data of male can be used as the reference data 420. Furthermore, for example, in a case where it is desired to compare the states of the last year and this year, the sensing data of last year on the same month and date when the sensing data 400 was acquired can be used as the reference data 420.

(Step S105)

The server 30 calculates the difference 402 by subtracting the reference data 420 selected in step S105 described above from the sensing data 400. At this time, the server 30 may perform normalization on the difference 402 or may perform another statistical processing.

(Step S107)

The server 30 converts the difference 402 into the difference time (time conversion) by multiplying the difference 402 calculated in step S105 described above by a predetermined coefficient. Note that the interpretation of the progress of time of the user time 412 with respect to the difference 402 (magnitude relationship) of each sensing data 400 is as already described, for example, with reference to Table 1.

(Step S109)

The server 30 integrates a plurality of difference times time-converted in step S107 described above. In detail, starting from a predetermined start time set by the user or the like, a plurality of difference times obtained by the predetermined end time (for example, the present time) set by the user or the like is integrated to obtain the integration time 406.

(Step S111)

The server 30 calculates the user time 412 on the basis of the integration time 406 integrated in step S109 described above. In detail, the server 30 synthesizes the integration times 406 of the plurality of different types of sensing data 400 by processing on the basis of a predetermined formula to calculate the index (time difference) 408 related to the user. Moreover, the server 30 calculates the user time 412 by adding the calculated index 408 related to the user to the standard time 410.

(Step S113)

The server 30 presents to the user the integration time 406 obtained in step S109 described above as the index (time difference) 408 related to the user, or the index 408 related to the user, the user time 412, and the like obtained in step S111 described above. Note that the details of the presentation method in the present embodiment will be described later.

As described above, according to the present embodiment, it is possible to provide the user time 412 for the user according to how the user feels the flow of time on the basis of the amount of exercise and the amount of load due to the user's action. Moreover, according to the present embodiment, since the amount of exercise and the amount of load of the user to be presented are replaced with the time point or time interval that is familiar to the user on a daily basis, as compared with the case where the amount of exercise or the like is directly presented, the user can easily understand its own state and the like. As a result, according to the present embodiment, it is possible to arouse the action change of the user based on the above understanding.

### 2.5. Setting of Start Time According to the Embodiment of the Present Disclosure

Next, an example of setting a range for integration of the difference time and, in detail, setting a start time for starting the integration will be described. In the present embodiment, various times can be set for the start time.

### Example 1

For example, in the present embodiment, it may be possible to assume that, when the user goes to bed, the difference between the standard time 410 and the user time 412 is reset, and when the user wakes up and performs activities, the difference between the standard time 410 and the user time 412 occurs. In a case where such assumption is made, in the present embodiment, the time when the user wakes up is set as the start time. Note that the time when the user wakes up can be detected by the motion sensor unit 124 of the wearable device 10.

### Example 2

Furthermore, for example, in the present embodiment, it may be possible to assume that the difference between the standard time 410 and the user time 412 always occurs even when the user goes to bed or performs activities. In a case where such assumption is made, in the present embodiment, the start of the wearable device 10 itself may be set as the start time, and the integration of the difference time may be continued while the wearable device 10 is running.

### Example 3

Furthermore, in the present embodiment, a time specified by the user, for example, 12:00 at the standard time 410 or the like can be set as the start time.

Moreover, in the present embodiment, the start time, the end time, the reset timing of the integration time 406, and the like can be appropriately changed by the user. Note that the above-mentioned example is shown as an example of the setting of the present embodiment, that is, the present embodiment is not limited to these examples.

### 2.6. Presentation Method According to the Embodiment of the Present Disclosure

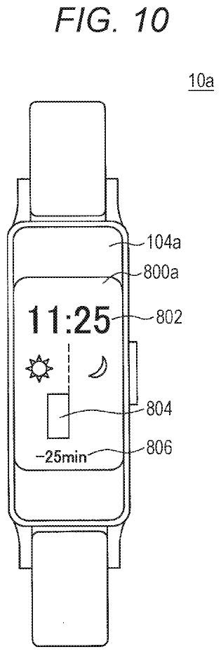

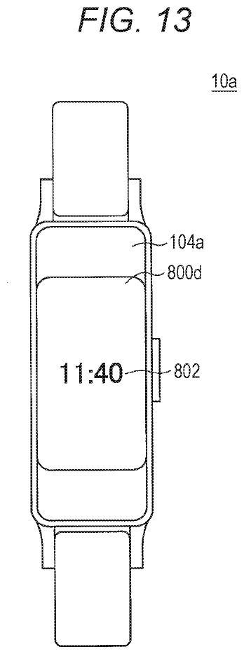

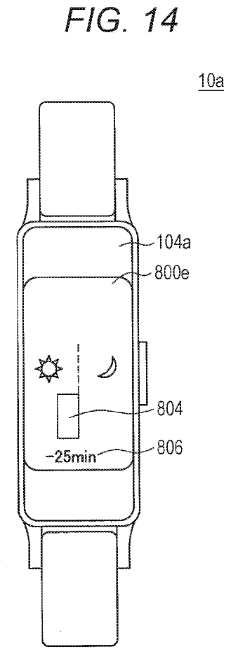

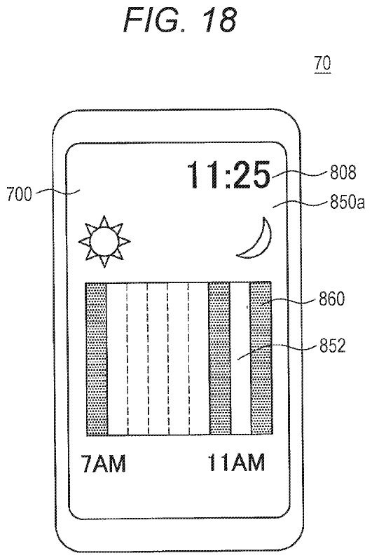

Next, the details of the presentation method according to the embodiment of the present disclosure will be described with reference to FIGS. 10 to 20. FIGS. 10 to 17 are explanatory diagrams for explaining an example of display screens 800a to 800h according to the embodiment of the present disclosure, and FIGS. 18 to 20 are explanatory diagrams for explaining an example of display screens 850a to 850c according to the embodiment of the present disclosure.

(First Presentation Method)

The first presentation method is a mode in which the user time 412 of the present time is presented according to the situation of the user on the day. Note that, in the first presentation method, the user time 412 and the like are only presented to the user, and the action or the like that the user should take on the day is not proposed to the user. That is, in the first presentation method, the user itself is expected to voluntarily take an appropriate action by referring to the user time 412 or the like.

In the present embodiment, for example, as shown in FIG. 10, the user time 412 is presented to the user by the display screen 800a displayed on the display unit 104a of the bracelet (wristband) type wearable device 10a. In detail, the display screen 800a can include, for example, a user time display 802 indicating the user time 412, an integration time graphic display 804 indicating the integration time 406 calculated as the index (time difference) 408 related to the user by the length of a bar graph, and an integration time display 806 indicating the integration time 406 described above. Note that, in the present embodiment, the integration time graphic display 804 and the integration time display 806 may indicate the integration time including a synthesized integration time 406 as the index 408 related to the user, or may indicate an unsynthesized integration time 406 obtained from one type of sensing data 400. For example, the integration time graphic display 804 indicates that the user time 412 is later than the standard time 410 as it extends to the left in the drawing and that the user time 412 is earlier than the standard time 410 as it extends to the right in the drawing. By looking at such a display screen 800a, for example, since the user time 412 is 25 minutes later than the standard time 410, the user would think that “Today, I can spend a relaxing time from the morning. I hope I can try little harder until noon”, and will increase the processing speed of business.

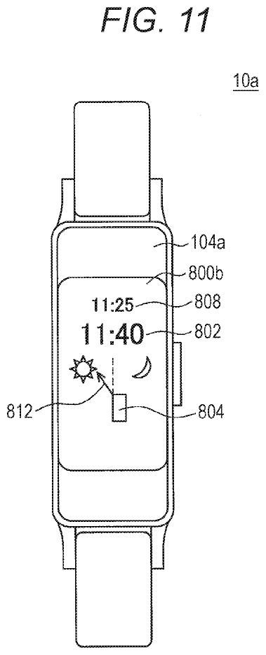

Furthermore, in the present embodiment, for example, as shown in FIG. 11, the user time 412 may be presented to the user by the display screen 800b displayed on the display unit 104a. In detail, the display screen 800b can include, for example, a user time display 802, a standard time display 808 indicating the standard time 410, and an integration time graphic display 804. By looking at such a display screen 800b, for example, since the user time 412 is 15 minutes faster than the standard time 410, the user would think that “Today, I was busy from the morning. I want to have lunch early today”, and will have lunch early.

Note that, as shown in FIG. 11, the display screen 800b may include a tendency display 812 having an arrow shape. The tendency display 812 indicates the progress of the user time 412 with respect to the standard time 410 in the latest predetermined time (for example, the latest 10 minutes). Specifically, for example, in a case where the tendency display 812 is tilted to the left in the drawing, it indicates that the user time 412 is later than the standard time 410 in the latest predetermined time, and in a case where the tendency display 812 is tilted to the right in the drawing, it indicates that the user time 412 is faster than the standard time 410 in the latest predetermined time.

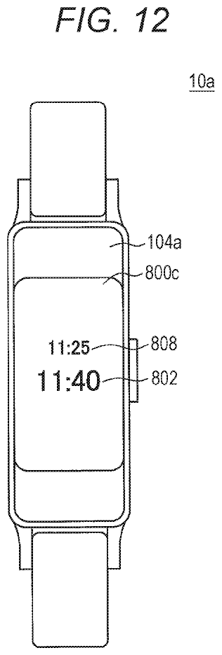

In the present embodiment, as shown in FIGS. 12 to 14, information may be presented to the user by displaying the user time display 802, the integration time graphic display 804, the integration time display 806, and the standard time display 808 in various combinations, and the form of the display screen 800 is not particularly limited. Furthermore, the user may operate the button 102a (see FIG. 7) to switch, for example, the display between the user time 412 and the standard time 410, or may switch the display between the integration time 406 and the standard time 410.

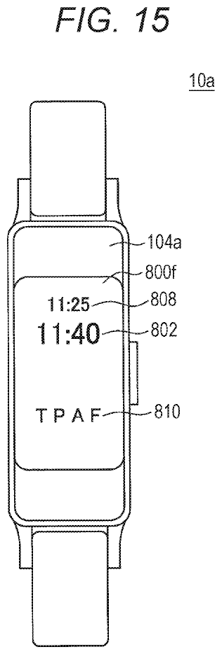

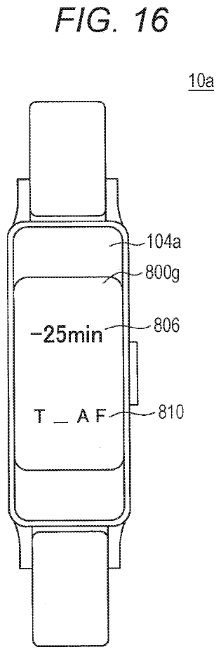

Furthermore, in the present embodiment, as shown in FIGS. 15 and 16, the type of sensing data used when calculating the user time 412 and the like may be presented to the user. For example, as shown in FIG. 15, the type of sensing data used for calculating the user time 412 and the like may be presented by a type display 810 included in the display screen 800f displayed on the display unit 104a. In detail, the display screen 800f can include, for example, a user time display 802, a standard time display 808 indicating the standard time 410, and the type display 810. The type display 810 displays the type of sensing data used when calculating the user time 412 and the like to the user by displaying various alphabets (corresponding, for example, to T: body temperature, P: pulse rate, A: acceleration, F: step count). In the example of FIG. 15, “T, P, A, F” is displayed, presenting that the user time 412 has been calculated using the sensing data 400 of body temperature, pulse rate, acceleration, and step count.

Furthermore, in the example of FIG. 16, “T, _, A, F” is displayed, that is, “P” is not displayed, presenting that the user time 412 has been calculated using the sensing data 400 of body temperature, acceleration, and step count, excluding pulse rate.

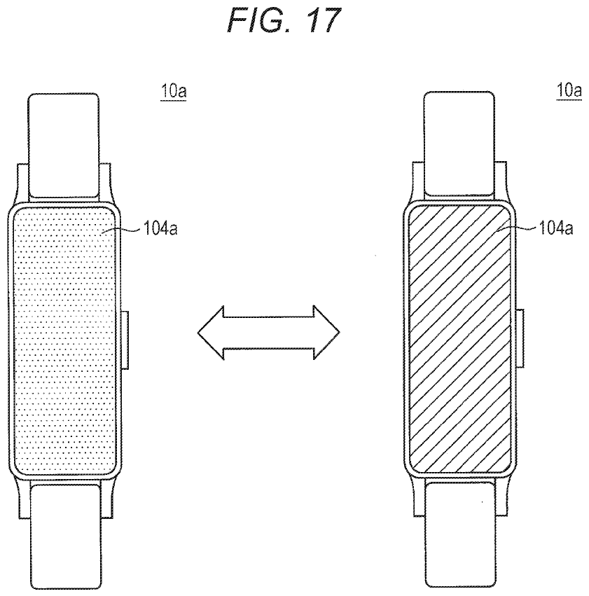

Moreover, in the present embodiment, as shown in FIG. 17, by switching the color, brightness, or pattern of the display unit 104a, the index 408 related to the user, which is the progress of the user time 412 with respect to the standard time 410, may be presented. For example, in a case where the display unit 104a has a bright color, it indicates that the user time 412 is later than the standard time 410, and in a case where the display unit 104a has a dark color, it indicates that the user time 412 is faster than the standard time 410. Moreover, in the present embodiment, the progress of the user time 412 with respect to the standard time 410 may be presented by a sound, a vibration pattern (for example, difference in vibration pattern), or the like.

(Second Presentation Method)

The second presentation method is a mode in which the progress (transition) of the user time 412 in a past predetermined period (for example, one day, several days, week, month, year) is presented. In the second presentation method, the user time 412 at one point at the present time is not presented as in the first presentation method, but by presenting the progress of the user time 412 over a wide period, information for considering the activities of the user and the like from more angles is presented. Then, in the second presentation method, it is expected that the actions to be performed by the users in the future and the quality of the actions itself will be changed by providing such multifaceted information. Note that, in the second presentation method, the server 30 performs preferable comparison by expanding the period for which the progress of the user time 412 is calculated, and it is preferable to change the reference data 420 used for calculation or the like to data different from that of the first presentation method.

For example, in the present embodiment, as shown in FIG. 18, the progress of the user time 412 of the day can be presented to the user by the display screen 850a displayed on the display unit 700 of the user terminal 70 including a smartphone. In detail, the display screen 850a includes, for example, a standard time display 808 indicating the current standard time 410 and a progress display 852 indicating the progress of the user time 412. The progress display 852 includes, for example, nine bands 860 that are obtained by dividing the time from 7:00 to 11:00 into nine hours and correspond to each of the divided hours. Moreover, the progress display 852 displays the progress of the user time 412 at each time with the color, pattern, or the like of the corresponding band 860. For example, in a case where the band 860 is shown in a bright color, it indicates that the progress of the user time 412 is slower than the standard time 410 at that time, and in a case where the band 860 is shown in a dark color, it indicates that the progress of the user time 412 is faster than the standard time 410.

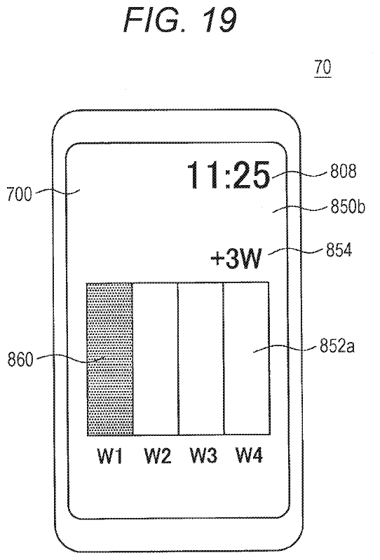

Furthermore, in the present embodiment, for example, as shown in FIG. 19, the progress of the user time 412 of one month may be presented to the user by the display screen 850b displayed on the display unit 700. In detail, the display screen 850b includes, for example, a standard time display 808 indicating the current standard time 410, a progress display 852a indicating the progress of the user time 412, and an index display 854 indicating an index of the tendency of the progress of the user time 412 of one month. The progress display 852a includes, for example, four bands 860 that are obtained by dividing the most recent month into four hours (weeks) and correspond to each week. Moreover, the progress display 852a displays the progress of the user time 412 of each week with the color, pattern, or the like of the corresponding band 860. Moreover, the index display 854 displays an index obtained by subtracting the number of progress fast weeks from the number of progress slow weeks of the user time 412 as an index indicating the tendency of the progress of user time 412 of the most recent month.

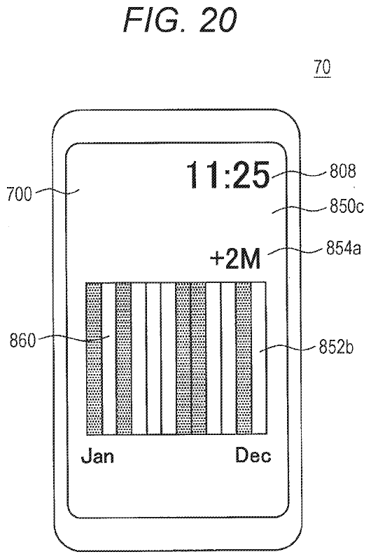

Moreover, in the present embodiment, for example, as shown in FIG. 20, the progress of the user time 412 of one year may be presented to the user by the display screen 850c displayed on the display unit 700. In detail, the display screen 850c includes, for example, a standard time display 808 indicating the current standard time 410, a progress display 852a indicating the progress of the user time 412, and an index display 854a indicating an index of the tendency of the progress of the user time 412 of one year. The progress display 852b includes, for example, twelve bands 860 that are obtained by dividing the most recent year into twelve hours (months) and correspond to each month. Moreover, the progress display 852b displays the progress of the user time 412 of each month with the color, pattern, or the like of the corresponding band 860. Moreover, the index display 854a displays an index obtained by subtracting the number of progress fast months from the number of progress slow months of the user time 412 as an index indicating the tendency of the progress of user time 412 of the most recent year.

As described above, in the present embodiment, the user can easily understand the user time 412 and the like by presenting the user time 412 and the like in a form that the user can intuitively understand. Moreover, according to the present embodiment, it is possible to arouse the action change of the user on the basis of the above understanding. Note that the examples shown in FIGS. 10 to 20 are shown as examples of the display screens 800 and 850 of the present embodiment, i.e., the display screens 800 and 850 according to the present embodiment are not limited to the examples shown in FIGS. 10 to 20.

### 2.7. Timing of Presentation According to the Embodiment of the Present Disclosure

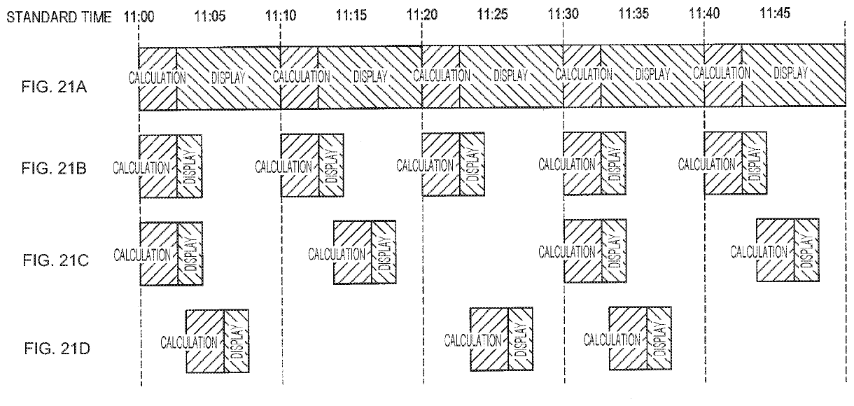

Next, the timing of presenting the user time 412 according to the present embodiment will be described with reference to FIG. 21. FIG. 21 is an explanatory diagram for explaining an example of a display timing according to the present embodiment. As shown in FIG. 21, in the present embodiment, various forms can be selected for the timing of presentation (display) of the user time 412 and the like.

In the present embodiment, for example, as shown in (a) of FIG. 21, the display of the user time 412 or the like is constantly continued, and the display described above may be updated at the timing when the calculation processing for the user time 412 is performed.

Furthermore, in the present embodiment, for example, as shown in (b) of FIG. 21, after the user time 412 is calculated, the user time 412 or the like may be displayed for a predetermined time (for example, one minute).

Furthermore, in the present embodiment, for example, as shown in (c) of FIG. 21, the user time 412 or the like may be calculated automatically every time (for example, 15 minutes) set by the user, and then the user time 412 or the like may be displayed for a predetermined time (for example, one minute).

For example, as shown in (d) of FIG. 21, in a case where the act of the user watching at the display unit 104a (see FIG. 4) of the bracelet type wearable device 10a is detected, the user time 412 or the like may be calculated automatically. Note that, in the present embodiment, the user's act of watching can be detected, for example, by detecting the user's tapping operation on the display unit 104a or by performing detection from the acceleration of the user's arm. Then, after the calculation, the display unit 104a may display the user time 412 or the like for a predetermined time (for example, one minute).

Furthermore, the user time 412 may be displayed only in a case where when the user time 412 is calculated for each predetermined time set in advance and the progress of the obtained user time 412 is significantly changed (for example, in a case where a change equal to or greater than the predetermined threshold value as compared with the progress of the user time 412 calculated previously is detected).

As described above, in the present embodiment, the timing of presenting the user time 412 and the like can be set to various forms. Therefore, it is possible to present information such as the user time 412 and the like at the request of the user and suppress an increase in power consumption by the presentation of the information. Note that the example shown in FIG. 21 is shown as an example of the timing of presentation of the present embodiment, i.e., the timing of presentation according to the present embodiment is not limited to the example shown in FIG. 21.

### 2.8. Timing of Calculation According to the Embodiment of the Present Disclosure

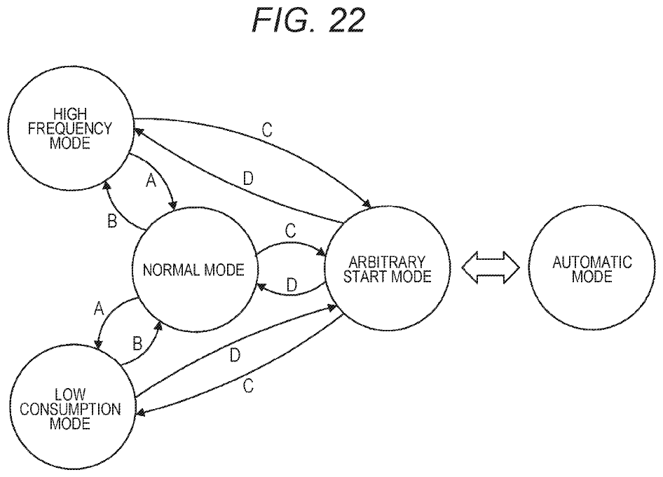

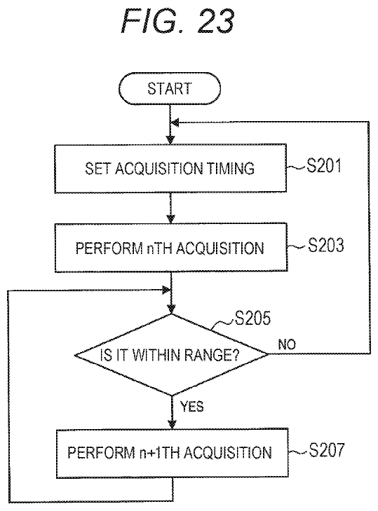

Next, the timing of calculation of the index 408 related to the user, the user time 412, and the like according to the present embodiment will be described with reference to FIGS. 22 and 23. FIG. 22 is an explanatory diagram for explaining an example of the transition of the calculation mode according to the present embodiment, and FIG. 23 is a flowchart showing an example of the information processing method of the automatic mode according to the embodiment of the present indication.

In the present embodiment, the timing of acquisition of the sensing data 400, the timing of calculation of the index 408 related to the user or the user time 412, and the like can be selected and changed appropriately according to the power consumption of the wearable device 10 and the like or the type of the sensing data 400 to be acquired.

For example, in the present embodiment, the calculation mode can be appropriately changed according to the user's settings, the power consumption of the wearable device 10, and the like. In the present embodiment, for example, five calculation modes can be set as shown in Table 2 below.

TABLE 2

| Calcu- | Sensing data acquisition timing | | | | Calcu- |
|---|---|---|---|---|---|
| lation | Body | | | | lation |
| mode | temperature | Pulse rate | Acceleration | Step count | timing |
| High | Four | Four | Constant | Constant | Four |
| frequency | times/ | times/ | acquisition | acquisition | times/ |
| mode | minute | minute | | | minute |
| Normal | Once/five | Once/five | Constant | Constant | Once/five |
| mode | minutes | minutes | acquisition | acquisition | minutes |
| Low | Once/five | Once/thirty | Constant | Constant | Once/ |
| con- | minutes | minutes | acquisition | acquisition | thirty |
| sumption | | | | | minutes |
| mode | | | | | |
| Arbitrary | At start | At start | Constant | Constant | At start |
| start mode | | | acquisition | acquisition | |
| Automatic | Arbitrary | Arbitrary | Constant | Constant | Arbitrary |
| mode | setting | setting | acquisition | acquisition | setting |

Note that the example shown in Table 2 is shown as an example of the calculation mode of the present embodiment. That is, the calculation mode and the setting conditions in each calculation mode according to the present embodiment are not limited to the example shown in Table 2.

Moreover, in the present embodiment, in a case where a predetermined condition is satisfied, the above calculation mode may be transitioned as shown in FIG. 21. For example, by transitioning to a low consumption mode, it is possible to suppress an increase in power consumed when acquiring sensing data about the pulse wave, and as a result, the wearable device 10 can be activated for a long period of time. Furthermore, for example, in a case where the same type of sensing data 400 changes significantly, the accuracy of the calculated user time 412 can be improved by transitioning the calculation mode to a high frequency mode according to the change.

Note that conditions A to D in FIG. 21 are, for example, as described below.

A: In a case where the difference (difference rate) between the previously acquired sensing data 400 and the currently acquired sensing data 400 is within a predetermined range (for example, within 10%).

B: In a case where the difference (difference rate) between the previously acquired sensing data 400 and the currently acquired sensing data 400 is out of a predetermined range (for example, 10% or more).

C: In a case where an instruction to acquire sensing data 400 is received from the user

D: In a case where the sensing data 400 is acquired

Note that the example shown in FIG. 21 is shown as an example of the transition of the calculation mode of the present embodiment. That is, the transition of the calculation mode and the conditions of the transition according to the present embodiment are not limited to FIG. 21 and the aforementioned conditions.

Furthermore, in the present embodiment, in the above automatic mode, the timing of acquisition of individual sensing data 400 may be changed only in a case where the difference (difference rate) between the previously acquired sensing data 400 and the currently acquired sensing data 400 is out of the predetermined range, or the like. In detail, for example, the acquisition interval is reduced only for the sensing data 400 having a large change width, and the acquisition interval so far is maintained for the sensing data 400 having a small change width. In this way, in the automatic mode, it is possible to improve the accuracy of the calculated user time 412 while suppressing the increase in power consumption.

More specifically, for example, as shown in FIG. 23, the automatic mode according to the present embodiment includes a plurality of steps from step S201 to step S207. The details of each step included in the automatic mode according to the present embodiment will be described below.

(Step S201)

The server 30 sets the acquisition timing (time interval) for each sensing data 400.

(Step S203)

The server 30 acquires the sensing data 400 on the basis of the setting in step S201 (nth acquisition).

(Step S205)

The server 30 compares the sensing data 400 acquired in n−1th time with the sensing data 400 acquired in step S203 described above, and determines whether the difference is within a predetermined range. The server 30 proceeds to step S207 when it is within the predetermined range, and returns to step S201 when it is out of the predetermined range. Then, in the returned step S201, the server 30 sets, on the basis of a predetermined rule, for example, the time interval related to the timing of acquisition of the corresponding sensing data 400 to be short.

(Step S207)

The server 30 acquires each sensing data 400 on the basis of the acquisition timing set first in step S201 (n+1th acquisition).

As described above, in the automatic mode, by the above processing, it is possible to improve the accuracy of the calculated user time 412 while suppressing the increase in power consumption.

### 2.9. Selection of the Reference Data 420 According to the Embodiment of the Present Disclosure

Next, the details of the selection of the reference data 420 according to the present embodiment will be described. In the present embodiment, the reference data 420 is preferably selected on the basis of the processing described below in order to calculate the user time 412 with higher accuracy. In detail, in the following processing, the reference data 420 is changed in a case where the calculated user time 412 has a significant difference from the previously calculated user time 412. By doing so, it is possible to select more suitable reference data 420 for calculating the user time 412 and the like with high accuracy.

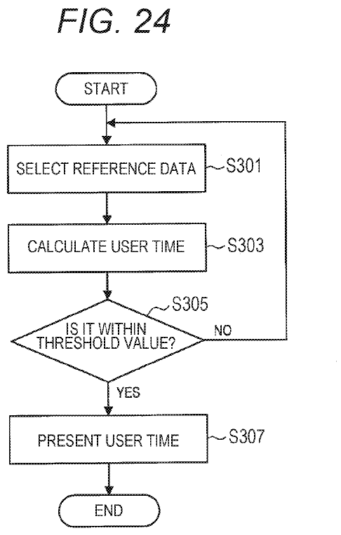

An example of the processing for selecting the reference data 420 in the present embodiment will be described with reference to FIG. 24. FIG. 24 is a flowchart showing an example of processing for selecting the reference data 420 according to the present embodiment. As shown in FIG. 24, the processing for selection according to the present embodiment includes a plurality of steps from step S301 to step S307. The details of each step will be described below.

(Step S301)

The server 30 selects the reference data 420 according to the user's attributes or the user's settings.

(Step S303)

The server 30 calculates the user time 412 on the basis of the selection in step S301 described above.

(Step S305)

The server 30 compares the user time 412 calculated previously with the user time 412 calculated in step S303 described above, and determines whether the difference is within a predetermined range. The server 30 proceeds to step S307 when it is within the predetermined range, and returns to step S301 when it is out of the predetermined range. Then, in step S301 to which the processing has returned, the server 30 selects the reference data 420 to be used for comparison with the sensing data 400 on the basis of a predetermined rule.

For example, in a case where the reference data 420 selected earlier is the sensing data obtained by smoothing a plurality of sensing data of the last three days from the day when the sensing data 400 was acquired, the server 30 newly selects the sensing data obtained by smoothing the plurality of sensing data of the last 5 days from the day when the sensing data 400 was acquired.

(Step S307)

The server 30 presents the user with the user time 412 calculated in step S303.

According to the present embodiment, by performing the above processing, it is possible to select more suitable reference data 420 for calculating the user time 412 and the like with high accuracy.

### 2.10. Feedback Processing of User Evaluation According to the Embodiment of the Present Disclosure

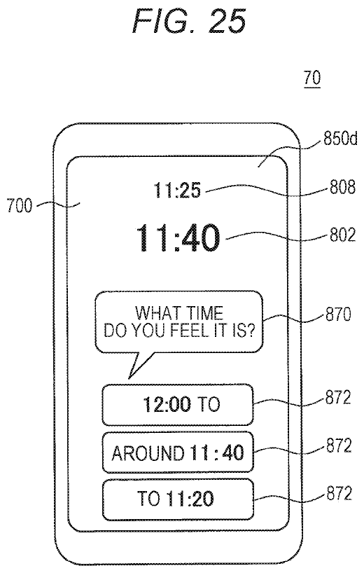

By the way, in the present embodiment, in order to calculate the user time 412 that is closer to the user's actual feeling, the user may perform evaluation and the evaluation may be fed back to the calculation of the user time 412. The feedback processing of the user evaluation according to the present embodiment will be described below with reference to FIG. 25. FIG. 25 is an explanatory diagram for explaining an example of the display screen 850d according to the present embodiment.

In detail, when presenting the user time 412 to the user, in order to obtain the user's evaluation with respect to the user time 412, the display screen 850d shown in FIG. 25 may be displayed for the user. For example, the display screen 850d displayed on the display unit 700 of the user terminal 70 including a smartphone includes a user time display 802 indicating the user time 412 and a standard time display 808 indicating the current standard time 410. Moreover, the display screen 850d includes a window 870 for asking the user for evaluation and a window 872 for the user to answer. Specifically, the window 870 is a window that asks the user for the evaluation of the user time 412 displayed for the user, for example, “What time do you feel it is?”. Furthermore, the window 872 is a window in which the evaluation can be input by the user performing an operation of selecting each window. For example, the user can perform an operation on any of “12:00˜(after 12:00)”, “around 11:40”, and “˜11:20 (before 11:20)” shown in each window 872 as options to input the evaluation with respect to the user time 412. Note that, in the present embodiment, the user's evaluation input may be sound input, and furthermore, the evaluation for the index 408 related to the user or the like instead of the user time 412 may be acquired.

Then, on the basis of such an evaluation result, the server 30 can set the user time 412 to a time closer to the actual feeling of the user by, for example, changing the coefficients a to e (weighting) described above.

Moreover, the server 30 may machine-learn the evaluation tendency according to each attribute by associating the evaluation tendency of each user obtained in this way with the attribute information of each user. Then, the server 30 may use the tendency obtained by machine learning when calculating the user time 412 of another user (for example, setting of the values of the coefficients a to e).

### 2.11. Example of Using a User Interface According to the Embodiment of the Present Disclosure

An example of using the user interface in a case where the wearable device 10 is the bracelet type wearable device 10a will be described below. As an example of using the user interface according to the present embodiment, the operation on the button 102a of the bracelet type wearable device 10a, the tap operation on the surface of the bracelet type wearable device 10a, and the operation of the corresponding bracelet type wearable device 10a are shown in Table 3 below.

TABLE 3

| | Operation on button 102a | | Tap operation | |
|---|---|---|---|---|
| | Short press | Long press | Single tap | Double tap |
| Case 1 | Standard time, | Sleep. Power | Tapped user | Tapped user |
| | user time, | is turned off | time is | time is stored |
| | pulse rate, | by long press | stored. | separately |
| | step count, | of ten seconds | | from single |
| | and the like | or more. | | tap. |
| | are switched | | | |
| | and displayed. | | | |
| Case 2 | On/off of | Sleep. Power | Standard time | Tapped user |
| | screen display | is turned off | and user time | time is |
| | is switched. | by long press | are switched | stored. |
| | | of ten seconds | and displayed. | |
| | | or more. | | |
| Case 3 | Standard time | Reset of user | On/off of | Standard time |
| | and user time | time. Power is | screen display | and user time |
| | are switched | turned off by | is switched. | are switched |
| | and displayed. | long press of | | and displayed. |
| | | ten seconds or | | |
| | | more. | | |

Note that, in the present embodiment, the tap operation can be detected by the acceleration sensor 120c of the motion sensor unit 124. Furthermore, in the present embodiment, by storing the user time 412 by the user's operation, the stored user time 412 can be used for future calculation of the user time 412 or verification of the calculation result.

Note that the example shown in Table 3 is an example of using the user interface of the present embodiment, and the example of using the user interface according to the present embodiment is not limited to Table 3.

### 3. Examples According to the Embodiment of the Present Disclosure

The details of the information processing method in the embodiment of the present disclosure have been described above. Next, an example of information processing according to the embodiment of the present disclosure will be described more specifically while showing specific examples. Note that the examples shown below are merely an example of information processing according to the embodiment of the present disclosure, and the information processing method according to the embodiment of the present disclosure is not limited to the examples described below.

### 3.1. Example 1

For example, an example will be described in a case where the user time 412 is 11:00 pm even though the standard time 410 is 9:00 pm. The user provided with such user time 412 will be aware that there is a possibility that the user is tired from busy working, and takes an action of going to bed earlier than 11:00 pm in the standard time 410 even though the user usually goes to bed at 11:00 pm in the standard time 410.

That is, according to the present example, the provided user time 412 allows the user to confirm the advance/lag of the user's own user time 412 before going to bed, and it is possible to arouse an action of going to bed for the user at a suitable timing.

### 3.2. Example 2

Furthermore, the embodiment of the present disclosure can also be used to arouse an action of maintaining a suitable sleep time for the user. In Example 2, in a case where the sensing data 400 obtained by the pulse wave sensor 120b or the like indicates that the user is sleeping for a longer time or the sleep depth is deeper than the reference value, it is assumed that the progress of time of the user time 412 is slower than the progress of time of the standard time 410.

For example, an example will be described in a case where the user time 412 is 6:00 am even though the standard time 410 is 8:00 am. The user provided with such user time 412 will be aware that there is a possibility that the user does not have sufficient sleeping, and further continue sleeping to a time later than 8:00 am in the standard time 410 even though the user usually goes to bed at 8:00 am in the standard time 410.

That is, according to the present example, the provided user time 412 allows the user to confirm the advance/lag of the user's own user time 412 before waking up, and it is possible to arouse an action of maintaining a suitable sleep time for the user.

As described above, according to the user time 412 provided by the present embodiment, the user can easily understand the pace (progress) of the user's own time due to his/her past activities. As a result, according to the present embodiment, when the action change of the user can be aroused, and eventually when the action according to the user time 412 can be appropriately taken, it can lead to the maintenance of the health of the user.

### 4. Conclusion

As described above, according to the above-described embodiment of the present disclosure, it is possible to provide the user time 412 for the user according to how the user feels the flow of time.

Moreover, according to the present embodiment, by providing the user time 412, it is possible to easily understand the difference in the current state from a suitable state, and therefore the action change of the user can be aroused. Furthermore, in the present embodiment, since the user time 412 is presented at the time point and time interval which are commonly recognizable indexes, the user or another user can easily comprehend the physical condition of the user and the like. Moreover, by using the user time 412, it becomes easy to understand the tendency of the state of a plurality of users (crowds).

Furthermore, it is difficult to comprehend what kind of state the user is showing from the numerical values such as heart rate, but it is easy to comprehend what kind of state the user is showing according to the user time 412 replaced with the time point or time interval that is familiar to the user on a daily basis. Moreover, even when various biological information is synthesized, it is possible to easily comprehend what kind of state the user is in by converting it to the user time 412.

Furthermore, in the above-described embodiment, the wearable device 10 may be a stand-alone apparatus by causing the wearable device 10 according to the present embodiment to have the function of the server 30.

### 5. Regarding Hardware Configuration

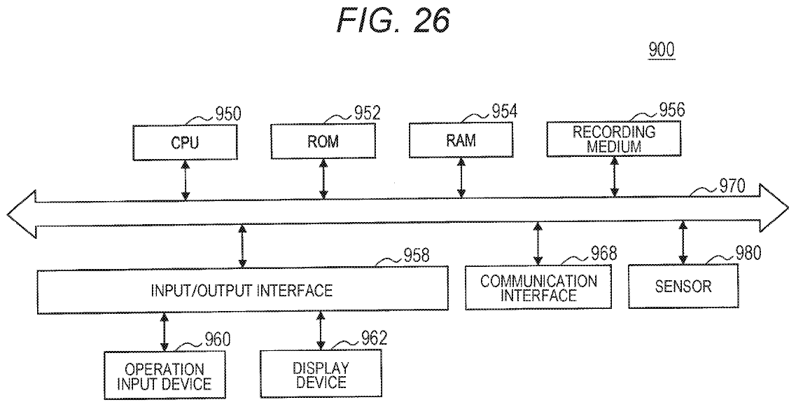

FIG. 26 is an explanatory diagram showing an example of a hardware configuration of the information processing apparatus 900 according to the present embodiment. Note that FIG. 26 shows an example of the hardware configuration of the server 30 described above in the information processing apparatus 900.

The information processing apparatus 900 includes, for example, a CPU 950, a ROM 952, a RAM 954, a recording medium 956, an input/output interface 958, and an operation input device 960. Moreover, the information processing apparatus 900 includes a display device 962, a communication interface 968, and a sensor 980. Furthermore, the information processing apparatus 900 connects the components with, for example, a bus 970 as a data transmission path.

(CPU 950)

The CPU 950 can function, for example, as the aforementioned main control unit 310 including one or two or more processors including an arithmetic circuit such as a central processing unit (CPU), various processing circuits, and the like, and controlling the entire information processing apparatus 900.

(ROM 952 and RAM 954)

The ROM 952 stores control data and the like such as a program, arithmetic parameters, or the like the CPU 950 uses. The RAM 954 functions as the above-mentioned storage unit 308, and temporarily stores, for example, a program executed by the CPU 950.

(Recording Medium 956)

The recording medium 956 functions as the above-mentioned storage unit 350, and stores, for example, various data such as data related to the information processing method according to the present embodiment, various applications, and the like. Here, examples of the recording medium 956 include a magnetic recording medium such as a hard disk and a non-volatile memory such as a flash memory. Furthermore, the recording medium 956 may be detachable from the information processing apparatus 900.

(Input/Output Interface 958, Operation Input Device 960, and Display Device 962)

The input/output interface 958 connects, for example, the operation input device 960, the display device 962, and the like. Examples of the input/output interface 958 include a universal serial bus (USB) terminal, a digital visual interface (DVI) terminal, a high-definition multimedia interface (HDMI) (registered trademark) terminal, and various processing circuits.

The operation input device 960 is provided in, for example, the information processing apparatus 900, and is connected to the input/output interface 958 inside the information processing apparatus 900. Examples of the operation input device 960 include buttons, direction keys, rotary selectors such as jog dials, touch panels, and combinations thereof.

The display device 962 is provided on, for example, the information processing apparatus 900, and is connected to the input/output interface 958 inside the information processing apparatus 900. Examples of the display device 962 include a liquid crystal display and an organic electro-luminescence (EL) display.

Note that, needless to say, the input/output interface 958 can also be connected to an external device such as an operation input device (for example, a keyboard or mouse) or a display device external to the information processing apparatus 900.

(Communication Interface 968)

The communication interface 968 is a communication means included in the information processing apparatus 900, and functions as the communication unit 306 that communicates wirelessly or by wire with an external apparatus such as the wearable device 10 or the user terminal 70 via the network 90 (or directly). Here, examples of the communication interface 968 include a communication antenna and a radio frequency (RF) circuit (wireless communication), an IEEE802.15.1 port and a transmission/reception circuit (wireless communication), an IEEE802.11 port and a transmission/reception circuit (wireless communication), or a local area network (LAN) terminal and a transmission/reception circuit (wired communication).

Heretofore, an example of the hardware configuration of the information processing apparatus 900 has been shown. Note that the hardware configuration of the information processing apparatus 900 is not limited to the configuration shown in FIG. 26. In detail, the components described above may be configured using universal members, or may be configured by hardware specific to the functions of the components. Such a configuration can be appropriately changed according to the technical level at the time of implementation.

For example, the information processing apparatus 900 may not include the communication interface 968 in a case where communication is performed with an external apparatus or the like via a connected external communication device or in a case of the configuration in which processing is performed in a stand-alone manner. Furthermore, the communication interface 968 may have a configuration capable of communicating with one or two or more external apparatuses by a plurality of communication schemes. Furthermore, the information processing apparatus 900 can be configured not to include, for example, the recording medium 956, the operation input device 960, the display device 962, or the like.

Furthermore, the information processing apparatus according to the present embodiment may be applied to a system including a plurality of apparatuses, which is premised on connection to a network (or communication between apparatuses), such as cloud computing. That is, the information processing apparatus according to the present embodiment described above can be realized as, for example, an information processing system that performs processing related to the information processing method according to the present embodiment by a plurality of apparatuses.

### 6. Supplement

Note that the embodiment of the present disclosure described above may include, for example, a program for causing a computer to function as the information processing apparatus according to the present embodiment, and a non-temporary tangible medium in which the program is recorded. Furthermore, the program may be distributed via a communication line (including wireless communication) such as the Internet.

Furthermore, each step in the processing of the above-described embodiment of the present disclosure does not necessarily have to be processed in the described order. For example, the order of the steps may be appropriately changed and processed. Furthermore, each step may be partially processed in parallel or individually instead of being processed in time series. Moreover, the processing method of each step does not necessarily have to be processed according to the described method, and may be processed by another method by another functional unit, for example.

The preferred embodiment of the present disclosure has been described above with reference to the accompanying drawings, while the technical scope of the present disclosure is not limited to the above examples. It is apparent that a person having normal knowledge in the technical field of the present disclosure may find various alterations and modifications within the scope of the technical idea stated in the claims, and it should be understood that they will naturally come under the technical scope of the present disclosure.

Furthermore, the effects described in the present specification are merely illustrative or exemplified effects, and are not limitative. That is, with or in the place of the above effects, the technology according to the present disclosure may achieve other effects that are clear to those skilled in the art from the description of the present specification.

Note that the configuration below also falls within the technical scope of the present disclosure.

(1)

An information processing apparatus including:

an information acquisition unit that acquires a temporal change in biological information from one or a plurality of biological information sensors worn by a user; and

a calculation unit that calculates a difference between a temporal change in first biological information in a first section and a temporal change in second biological information in a second section having a same time as the first section at predetermined time intervals and calculates a time difference with respect to a standard time.

(2)

The information processing apparatus according to (1), further including: a time calculation unit that calculates a time related to the user by adding the calculated time difference to the standard time.

(3)

The information processing apparatus according to (1) or

(2), in which the calculation unit calculates the time difference by converting the difference into time and integrating a plurality of the time-converted differences.

(4)

The information processing apparatus according to any one of (1) to (3), in which

the information acquisition unit acquires a temporal change in a plurality of pieces of the biological information of different types from a plurality of the different biological information sensors, and

the calculation unit calculates the time difference on the basis of the temporal change in the plurality of pieces of the biological information of the different types, which is weighted on the basis of the type of the biological information.

(5)

The information processing apparatus according to (4), further including:

an evaluation acquisition unit that acquires an evaluation for the time difference from the user, in which

the calculation unit performs weighting on the basis of the acquired evaluation.

(6)

The information processing apparatus according to any one of (1) to (5), in which the calculation unit selects the temporal change in the biological information used when calculating the time difference on the basis of reliability of each of the biological information.

(7)

The information processing apparatus according to any one of (1) to (6), in which the calculation unit selects the temporal change in the second biological information according to attributes of the user.

(8)

The information processing apparatus according to (7), in which

the temporal change in the second biological information includes the temporal change in a plurality of pieces of the biological information acquired in a plurality of the second sections and acquired from the biological information sensor worn by the user, the second section having a same time length as the first section in past of the first section.

(9)

The information processing apparatus according to (8), in which

the temporal change in the second biological information includes a temporal change obtained by smoothing temporal changes in a plurality of pieces of the biological information acquired in a plurality of the second sections and acquired from the biological information sensor worn by the user, the second section having the same time length as the first section in a period of a predetermined number of days satisfying a predetermined condition in a latest past in the first section.

(10)

The information processing apparatus according to (9), in which the calculation unit selects, as the predetermined condition, the temporal change in the second biological information having the second section having a same day of week as a day of week related to the first section.

(11)

The information processing apparatus according to (7), in which

the temporal change in the second biological information includes the temporal change in a plurality of pieces of the biological information acquired in a plurality of the second sections and acquired from the biological information sensor worn by another user other than the user, the second section having the same time length as the first section in past of the first section.

(12)

The information processing apparatus according to any one of (1) to (11), in which

the temporal change in the biological information is acquired by at least one of:

a beat sensor that detects heartbeat or pulse, a temperature sensor that detects skin temperature, a sweating sensor that detects sweating, a blood pressure sensor that detects blood pressure, a brain wave sensor that detects brain wave, a respiration sensor that detects respiration, a myoelectric potential sensor that detects myoelectric potential, and a blood oxygen concentration sensor that detects blood oxygen concentration, that are directly worn by a part of a body of the user, or

a motion sensor or position sensor that detects movement of the user.

(13)

The information processing apparatus according to (12), in which the motion sensor includes at least one of an acceleration sensor, a gyro sensor, or a geomagnetic sensor worn by the user.

(14)

The information processing apparatus according to (2), further including: a presentation unit that presents the calculated time difference to the user.

(15)

The information processing apparatus according to (14), in which the presentation unit displays the calculated time related to the user to the user.

(16)

The information processing apparatus according to (14), in which the presentation unit changes a color or a pattern to display the time difference.

(17)

The information processing apparatus according to any one of (1) to (16), in which the information acquisition unit or the calculation unit changes a timing of acquiring the temporal change in the first biological information or a timing of calculating the time difference according to power consumption of the biological information sensor and a state of the temporal change in the first biological information.

(18)

An information processing method including:

acquiring a temporal change in biological information from one or a plurality of biological information sensors worn by a user; and

calculating a difference between a temporal change in first biological information in a first section and a temporal change in second biological information in a second section having a same time as the first section at predetermined time intervals and calculating a time difference with respect to a standard time.

(19)

A program for causing a computer to execute:

a function of acquiring a temporal change in biological information from one or a plurality of biological information sensors worn by a user; and

a function of calculating a difference between a temporal change in first biological information in a first section and a temporal change in second biological information in a second section having a same time as the first section at predetermined time intervals and calculating a time difference with respect to a standard time.

### REFERENCE SIGNS LIST

1 Information processing system
10, 10a Wearable device
30 Server
70 User terminal
90 Network
100 Main body
102, 302 Input unit
102a Button
104, 304 Output unit
104a, 700 Display unit
106, 306 Communication unit
108, 308 Storage unit
110, 310 Main control unit
120 Sensor unit
120a Anti-surface temperature sensor
120b, 122 Pulse wave sensor (pulse wave sensor unit)
120c Acceleration sensor
120d Step count sensor
124 Motion sensor unit
150 Wristband
320 Sensing data acquisition unit
322 Evaluation acquisition unit
330 Processing unit
332 Index calculation unit
334 Time calculation unit
340 Output control unit
400, 400a, 400b, 400c, 400d Sensing data
402 Difference
406, 406a, 406b, 406c, 406d Integration time
408 Index
410 Standard time
412 User time
420 Reference data
600 Synthesis algorithm
800, 800a, 800b, 800c, 800d, 800e, 800f, 800g, 800h, 850, 850a,
850b, 850c, 850d Display screen
802 User time display
804 Integration time graphic display
806 Integration time display
808 Standard time display
810 Type display
812 Tendency display
852, 852a, 852b Progress display
854, 854a Index display
860 Band
870, 872 Window
900 Information processing apparatus
950 CPU
952 ROM
954 RAM
956 Recording medium
958 Input/output interface
960 Operation input device
962 Display device
968 Communication interface
970 Bus
980 Sensor

1. An information processing apparatus comprising:
an information acquisition unit that acquires a temporal change in biological information from one or a plurality of biological information sensors worn by a user; and
a calculation unit that calculates a difference between a temporal change in first biological information in a first section and a temporal change in second biological information in a second section having a same time as the first section at predetermined time intervals and calculates a time difference with respect to a standard time.

2. The information processing apparatus according to claim 1, further comprising: a time calculation unit that calculates a time related to the user by adding the calculated time difference to the standard time.

3. The information processing apparatus according to claim 1, wherein the calculation unit calculates the time difference by converting the difference into time and integrating a plurality of the time-converted differences.

4. The information processing apparatus according to claim 1, wherein
the information acquisition unit acquires a temporal change in a plurality of pieces of the biological information of different types from a plurality of the different biological information sensors, and
the calculation unit calculates the time difference on a basis of the temporal change in the plurality of pieces of the biological information of the different types, which is weighted on a basis of the type of the biological information.

5. The information processing apparatus according to claim 4, further comprising:
an evaluation acquisition unit that acquires an evaluation for the time difference from the user, wherein
the calculation unit performs weighting on a basis of the acquired evaluation.

6. The information processing apparatus according to claim 1, wherein the calculation unit selects the temporal change in the biological information used when calculating the time difference on a basis of reliability of each of the biological information.

7. The information processing apparatus according to claim 1, wherein the calculation unit selects the temporal change in the second biological information according to attributes of the user.

8. The information processing apparatus according to claim 7, wherein
the temporal change in the second biological information includes the temporal change in a plurality of pieces of the biological information acquired in a plurality of the second sections and acquired from the biological information sensor worn by the user, the second section having a same time length as the first section in past of the first section.

9. The information processing apparatus according to claim 8, wherein
the temporal change in the second biological information includes a temporal change obtained by smoothing temporal changes in a plurality of pieces of the biological information acquired in a plurality of the second sections and acquired from the biological information sensor worn by the user, the second section having the same time length as the first section in a period of a predetermined number of days satisfying a predetermined condition in a latest past in the first section.

10. The information processing apparatus according to claim 9, wherein the calculation unit selects, as the predetermined condition, the temporal change in the second biological information having the second section having a same day of week as a day of week related to the first section.

11. The information processing apparatus according to claim 7, wherein
the temporal change in the second biological information includes the temporal change in a plurality of pieces of the biological information acquired in a plurality of the second sections and acquired from the biological information sensor worn by another user other than the user, the second section having the same time length as the first section in past of the first section.

12. The information processing apparatus according to claim 1, wherein
the temporal change in the biological information is acquired by at least one of:
a beat sensor that detects heartbeat or pulse, a temperature sensor that detects skin temperature, a sweating sensor that detects sweating, a blood pressure sensor that detects blood pressure, a brain wave sensor that detects brain wave, a respiration sensor that detects respiration, a myoelectric potential sensor that detects myoelectric potential, and a blood oxygen concentration sensor that detects blood oxygen concentration, that are directly worn by a part of a body of the user, or
a motion sensor or position sensor that detects movement of the user.

13. The information processing apparatus according to claim 12, wherein the motion sensor includes at least one of an acceleration sensor, a gyro sensor, or a geomagnetic sensor worn by the user.

14. The information processing apparatus according to claim 2, further comprising: a presentation unit that presents the calculated time difference to the user.

15. The information processing apparatus according to claim 14, wherein the presentation unit displays the calculated time related to the user to the user.

16. The information processing apparatus according to claim 14, wherein the presentation unit changes a color or a pattern to display the time difference.

17. The information processing apparatus according to claim 1, wherein the information acquisition unit or the calculation unit changes a timing of acquiring the temporal change in the first biological information or a timing of calculating the time difference according to power consumption of the biological information sensor and a state of the temporal change in the first biological information.

18. An information processing method comprising:
acquiring a temporal change in biological information from one or a plurality of biological information sensors worn by a user; and
calculating a difference between a temporal change in first biological information in a first section and a temporal change in second biological information in a second section having a same time as the first section at predetermined time intervals and calculating a time difference with respect to a standard time.

19. A program for causing a computer to execute:
a function of acquiring a temporal change in biological information from one or a plurality of biological information sensors worn by a user; and
a function of calculating a difference between a temporal change in first biological information in a first section and a temporal change in second biological information in a second section having a same time as the first section at predetermined time intervals and calculating a time difference with respect to a standard time.
